(19) United States
(12) Patent Application Publication
(10) Pub. No.: US 2008/0207722 A1
(43) Pub. Date: Aug. 28, 2008

(54) Glepp-1 Inhibitors in the Treatment of Autoimmune and/or Inflammatory Disorders

(75) Inventors: Agnes Bombrun, Chambesy (CH); Rob Hooft Van Huijsduijnen, Bernex (CH); Catherine Jorand-Lebrun, Contamine-Sarzin (FR); Pierre-Alain Vitte, Cranves-Sales (FR); Patrick Gerber, Etoy (CH)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: LABORATORIES SERONO SA, COINSINS, VAUD (CH)

(21) Appl. No.: 11/916,097

(22) PCT Filed: Jul. 14, 2006
(86) PCT No.: PCT/EP2006/064288

§ 371 Date: Nov. 30, 2007

Related U.S. Application Data
(60) Provisional application No. 60/706,365, filed on Aug. 8, 2005.

(30) Foreign Application Priority Data
Jul. 15, 2005 (EP) 05106547.2

(51) Int. Cl. A61K 31/415; A61K 31/19; A61K 31/404; A61P 29/00

(52) U.S. Cl. 514/404; 514/568; 514/418

(57) ABSTRACT

The present invention is related to the use of a Glepp-1 inhibitor for the manufacture of a medicament for the treatment of an autoimmune and/or an inflammatory disorder.

### FIELD OF THE INVENTION

The present invention is related to the use of Glepp-1 inhibitors for the manufacture of a medicament for the treatment of autoimmune and/or inflammatory disorders.

### BACKGROUND OF THE INVENTION

Protein-tyrosine phosphatases (PTPs) play an important role in the regulation of phosphorylation of proteins and represent the counterparts of kinases. Among classical PTPs, there are two types: (i) non-receptor or intracellular PTPs and (ii) receptor-like PTPs. Most intracellular PTPs contain one catalytic domain only, whereas most receptor-like enzymes contain two. The catalytic domain consists of about 250 amino acids (Niels Peter Hundahl Moller et al. Protein tyrosine phosphatases (PTPs) as drug targets: Inhibitors of PTP-1B for the treatment of diabetes; Current Opinion in Drug Discovery & Development 3(5), 527-540 (2000)).

Protein-tyrosine phosphatases have been shown to be involved in various disorders. For instance PTP1B is generally viewed as being related to the insulin signalling and therefore is believed to be involved in disorders like diabetes mellitus type II.

In Nature, Vol. 5, January 2005, Tomas Mustelin et al presented a summary of what is known in respect of protein-tyrosine phosphatases and the immune response: A mouse having a mutation in SHP1 has provided a first example of an autoimmune disease caused by a defect in a PTP. The same is true for CD45 mutants as well as PEP (the mouse ortholog of LYP).

GLEPP-1 (also known as Glomerular epithelial protein 1, PTP-U2, PTPRO, PTP-oc, PTP-BK, PTP-Φ; GenBank Accession U20489) was discovered as a gene whose expression in Leukemia cell line U937 is overexpressed by Phorbol ester. The gene is expressed in macrophages, kidney, brain (Seimiya and Tsuruo 1993) and B-cells (Aguiar, Yakushijin et al. 1999).

Glomerular epithelial protein 1 (Glepp-1) is a receptor tyrosine phosphatase present on apical cell surface of the glomerular podocyte. The podocyte (visceral glomerular epithelial cell) is one of the major cell types responsible for the maintenance of the structure and function of the glomerular filter. Water, ions and small molecules from blood easily cross the filter while the passage of larger blood proteins (e.g. albumin) is minimized. Glepp-1 has a single transmembrane domain, a single intracellular phosphatase domain and a large extracellular domain comprising fibronectin type III-like repeats

Glepp1 mutant mice are healthy and viable. These animals have only been studied for kidney morphology and functionality. While subtle morphological changes were observed, kidney functionality in the mutant mice is normal (Wharram, Goyal et al. 2000).

Work in macrophages shows that Glepp1 expression is regulated by CSF-1 (colony-stimulating factor). Glepp1 dephosphorylates paxillin and is required for cell motility (chemotaxis and extravasation; Pixley, Lee et al. 1995; Pixley, Lee et al. 2001).

Recent work shows that the Glepp1 gene is often hypermethylated and silenced in lung and colon cancers (Nori, Yin et al. 2004; Motiwala, Kutay et al. 2004). However, no mention is made of increased tumor incidence in the Glepp1 knockout mice (Wharram, Goyal et al. 2000).

### SUMMARY OF THE INVENTION

The present invention is related to the use of a Glepp-1 inhibitor for the manufacture of a medicament for the treatment of autoimmune and/or inflammatory disorders.

### DETAILED DESCRIPTION OF THE INVENTION

The following paragraphs provide definitions of the various chemical moieties that make up the compounds according to the invention and are intended to apply uniformly through-out the specification and claims unless an otherwise expressly set out definition provides a broader definition.

“Glepp-1” refers to Glomerular epithelial protein 1 and has the following synonyms: PTP-oc (rabbit), PTPRO, PTP-U2, PTP-U2L, PTP-U2S, PTP-phi.

“C₁-C₆-alkyl” refers to alkyl groups having 1 to 6 carbon atoms. This term is exemplified by groups such as methyl, ethyl, n-propyl, isopropyl, n-butyl, isobutyl, tert-butyl, n-pentyl, n-hexyl and the like.

“C₁-C₈-alkyl” refers to alkyl groups having 1 to 8 carbon atoms. This term is exemplified by groups such as methyl, ethyl, n-propyl, isopropyl, n-butyl, isobutyl, tert-butyl, n-pentyl, n-hexyl, n-heptyl, n-octyl, 2-ethylhexyl, 2-octyl, 3-octyl, isooctyl and the like.

“C₁-C₁₂-alkyl” refers to alkyl groups having 1 to 12 carbon atoms. This term is exemplified by groups such as methyl, ethyl, n-propyl, isopropyl, n-butyl, isobutyl, tert-butyl, n-pentyl, n-hexyl, n-heptyl, n-octyl, 2-ethylhexyl, 2-octyl, 3-octyl, isooctyl, n-nonyl, 2-nonyl, 3-nonyl, 4-nonyl, isononyl, n-decyl, isodecyl, 2-decyl, 3-decyl, n-undecyl, n-dodecyl and the like.

“C₆-C₁₅-alkyl” refers to alkyl groups having 6 to 15 carbon atoms. This term is exemplified by groups such as n-hexyl, 2-hexyl, 3-hexyl, n-heptyl, n-octyl, 2-ethylhexyl, 2-octyl, 3-octyl, isooctyl, n-nonyl, 2-nonyl, 3-nonyl, 4-nonyl, isononyl, n-decyl, isodecyl, 2-decyl, 3-decyl, n-undecyl, n-dodecyl and the like.

“C₇-C₁₅-alkyl” refers to alkyl groups having 7 to 15 carbon atoms. This term is exemplified by groups such as n-heptyl, n-octyl, 2-ethylhexyl, 2-octyl, 3-octyl, isooctyl, n-nonyl, 2-nonyl, 3-nonyl, 4-nonyl, isononyl, n-decyl, isodecyl, 2-decyl, 3-decyl, n-undecyl, n-dodecyl and the like.

“C₈-C₁₂-alkyl” refers to alkyl groups having 8 to 12 carbon atoms. This term is exemplified by groups such as n-octyl, 2-ethylhexyl, 2-octyl, 3-octyl, isooctyl, n-nonyl, 2-nonyl, 3-nonyl, 4-nonyl, isononyl, n-decyl, isodecyl, 2-decyl, 3-decyl, n-dodecyl and the like.

“Aryl” refers to an unsaturated aromatic carbocyclic group of from 6 to 14 carbon atoms having a single ring (e.g., phenyl) or multiple condensed rings (e.g, naphthyl). Preferred aryl include phenyl, naphthyl, phenantrenyl and the like.

“C₁-C₆-alkyl aryl” refers to C₁-C₆-alkyl groups having an aryl substituent, including benzyl, phenethyl and the like.

“Heteroaryl” refers to a monocyclic heteroaromatic, or a bicyclic or a tricyclic fused-ring heteroaromatic group. Particular examples of heteroaromatic groups include optionally substituted pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadia-zolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxa-zolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl or benzoquinolyl.

“C₁-C₆-alkyl heteroaryl” refers to C₁-C₆-alkyl groups having a heteroaryl substituent, including 2-furylmethyl, 2-thienylmethyl, 2-(1H-indol-3-yl)ethyl and the like.

“C₂-C₆-alkenyl” refers to alkenyl groups preferably having from 2 to 6 carbon atoms and having at least 1 or 2 sites of alkenyl unsaturation. Preferable alkenyl groups include ethenyl (—CH═CH₂), n-2-propenyl (allyl, —CH₂CH═CH₂) and the like.

“C₂-C₆-alkenyl aryl” refers to C₂-C₆-alkenyl groups having an aryl substituent, including 2-phenylvinyl and the like.

“C₂-C₆-alkenyl heteroaryl” refers to C₂-C₆-alkenyl groups having a heteroaryl substituent, including 2-(3-pyridinyl)vinyl and the like.

“C₂-C₁₂-alkenyl” refers to alkenyl groups preferably having from 2 to 12 carbon atoms and having at least 1 or 2 sites of alkenyl unsaturation. Preferable alkenyl groups include ethenyl (—CH═CH₂), n-2-propenyl (allyl, —CH₂CH═CH₂), a variety of butenyl isomers, crotonyl, isopentenyl, and the like.

“C₂-C₁₅-alkenyl” refers to alkenyl groups preferably having from 2 to 15 carbon atoms and having at least 1 or 2 sites of alkenyl unsaturation. Preferable alkenyl groups include ethenyl (—CH═CH₂), n-2-propenyl (allyl, —CH₂CH═CH₂), a variety of butenyl isomers, crotonyl, isopentenyl, and the like.

“C₂-C₆-alkynyl” refers to alkynyl groups preferably having from 2 to 6 carbon atoms and having at least 1-2 sites of alkynyl unsaturation, preferred alkynyl groups include ethynyl (—C≡CH), propargyl (—CH₂C≡CH), and the like.

“C₂-C₆-alkynyl aryl” refers to C₂-C₆-alkynyl groups having an aryl substituent, including phenylethynyl and the like.

“C₂-C₆-alkynyl heteroaryl” refers to C₂-C₆-alkynyl groups having a heteroaryl substituent, including 2-thienylethynyl and the like.

“C₂-C₁₂-alkynyl” refers to alkynyl groups preferably having from 2 to 12 carbon atoms and having at least 1-2 sites of alkynyl unsaturation, preferred alkynyl groups include ethynyl (—C≡CH), propargyl (—CH₂C≡CH), 6-heptynyl, 7-octynyl, 8-nonynyl, and the like.

“C₂-C₁₅-alkynyl” refers to alkynyl groups preferably having from 2 to 15 carbon atoms and having at least 1-2 sites of alkynyl unsaturation, preferred alkynyl groups include ethynyl (—C≡CH), propargyl (—CH₂C≡CH), 6-heptynyl, 7-octynyl, 8-nonynyl, and the like.

“C₃-C₈-cycloalkyl” refers to a saturated carbocyclic group of from 3 to 8 carbon atoms having a single ring (e.g., cyclohexyl) or multiple condensed rings (e.g., norbornyl). Preferred cycloalkyl include cyclopentyl, cyclohexyl, norbornyl and the like.

“C₁-C₆-alkyl cycloalkyl” refers to C₁-C₆-alkyl groups having a cycloalkyl substituent, including cyclohexylmethyl, cyclopentylpropyl, and the like.

“heterocycloalkyl” refers to a C₃-C₈-cycloalkyl group according to the definition above, in which 1 to 3 carbon atoms are replaced by hetero atoms chosen from the group consisting of O, S, NR, R being defined as hydrogen or C₁-C₆ alkyl. Preferred heterocycloalkyl include pyrrolidine, piperidine, piperazine, 1-methylpiperazine, morpholine, and the like.

“C₁-C₆-alkyl heterocycloalkyl” refers to C₁-C₆-alkyl groups having a heterocycloalkyl substituent, including 2-(1-pyrrolidinyl)ethyl, 4-morpholinylmethyl, (1-methyl-4-piperidinyl)methyl and the like.

“Carboxylic acid” refers to the group —C(O)OH.

“C₁-C₆-alkyl carboxy” refers to C₁-C₆-alkyl groups having a carboxy substituent, including 2-carboxyethyl and the like.

“Acyl” refers to the group —C(O)R where R includes H, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynyl heteroaryl”, “C₁-C₆-alkyl cycloalkyl”, “C₁-C₆-alkyl heterocycloalkyl”.

“C₁-C₆-alkyl acyl” refers to C₁-C₆-alkyl groups having an acyl substituent, including 2-acetylethyl and the like.

“Aryl acyl” refers to aryl groups having an acyl substituent, including 2-acetylphenyl and the like.

“Heteroaryl acyl” refers to hetereoaryl groups having an acyl substituent, including 2-acetylpyridyl and the like.

“C₃-C₈-(hetero)cycloalkyl acyl” refers to 3 to 8 membered cycloalkyl or heterocycloalkyl groups having an acyl substituent.

“Acyloxy” refers to the group —OC(O)R where R includes H, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C2-C₆-alkynyl heteroaryl”, “C₁-C₆-alkyl cycloalkyl”, “C₁-C₆-alkyl heterocycloalkyl”.

“C₁-C₆-alkyl acyloxy” refers to C₁-C₆-alkyl groups having an acyloxy substituent, including 2-(acetyloxy)ethyl and the like.

“Alkoxy” refers to the group —O—R where R includes “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C2-C6-alkynyl heteroaryl”, “C₁-C₆-alkyl cycloalkyl”, “C₁-C₆-alkyl heterocycloalkyl”.

“C₁-C₆-alkyl alkoxy” refers to C₁-C₆-alkyl groups having an alkoxy substituent, including 2-ethoxyethyl and the like.

“Alkoxycarbonyl” refers to the group —C(O)OR where R includes “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynyl heteroaryl”, “C₁-C₆-alkyl cycloalkyl”, “C₁-C₆-alkyl heterocycloalkyl”.

“C₁-C₆-alkyl alkoxycarbonyl” refers to C₁-C₆-alkyl groups having an alkoxycarbonyl substituent, including 2-(benzyloxycarbonyl)ethyl and the like.

“Aminocarbonyl” refers to the group —C(O)NRR′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynyl heteroaryl”, “C₁-C₆-alkyl cycloalkyl”, “C₁-C₆-alkyl heterocycloalkyl”.

“C₁-C₆-alkyl aminocarbonyl” refers to C₁-C₆-alkyl groups having an aminocarbonyl substituent, including 2-(dimethylaminocarbonyl)ethyl and the like.

“Acylamino” refers to the group —NRC(O)R′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynyl heteroaryl”, “C₁-C₆-alkyl cycloalkyl”, “C₁-C₆-alkyl heterocycloalkyl”.

“C₁-C₆-alkyl acylamino” refers to C₁-C₆-alkyl groups having an acylamino substituent, including 2-(propionylamino)ethyl and the like.

“Ureido” refers to the group —NRC(O)NR′R″ where each R, R′, R″ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynyl heteroaryl”, “C₁-C₆-alkyl cycloalkyl”, “C₁-C₆-alkyl heterocycloalkyl”, and where R′ and R″, together with the nitrogen atom to which they are attached, can optionally form a 3-8-membered heterocycloalkyl ring.

“C₁-C₆-alkyl ureido” refers to C₁-C₆-alkyl groups having an ureido substituent, including 2-(N′-methylureido)ethyl and the like.

“Carbamate” refers to the group —NRC(O)OR′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl carbamate” refers to C₁-C₆-alkyl groups having a carbamate substituent.

“Amino” refers to the group —NRR′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynyl heteroaryl”, “C₁-C₆-alkyl cycloalkyl”, “C₁-C₆-alkyl heterocycloalkyl”, and where R and R′, together with the nitrogen atom to which they are attached, can optionally form a 3-8-membered hetero-cycloalkyl ring.

“C₁-C₆-alkyl amino” refers to C₁-C₆-alkyl groups having an amino substituent, including 2-(1-pyrrolidinyl)ethyl and the like.

“Ammonium” refers to a positively charged group —N⁺RR′R″, where each R, R′, R″ is independently, “C₁-C₆-alkyl”, “C₂-C₆-alkenyl”, “C₂-C₆-alkynyl”, “C₃-C₈-cycloalkyl”, “heterocycloalkyl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynyl heteroaryl”, “C₁-C₆-alkyl cycloalkyl”, “C₁-C₆-alkyl heterocycloalkyl”, and where R and R′, together with the nitrogen atom to which they are attached, can optionally form a 3-8-membered heterocycloalkyl ring.

“C₁-C₆-alkyl ammonium” refers to C₁-C₆-alkyl groups having an ammonium substituent, including 2-(1-pyrrolidinyl)ethyl and the like.

“Halogen” refers to fluoro, chloro, bromo and iodo atoms.

“Sulfonyloxy” refers to a group —OSO₂—R wherein R is selected from H, “C₁-C₆-alkyl”, “C₁-C₆-alkyl” substituted with halogens, e.g., an —OSO₂—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynyl heteroaryl”, “C₁-C₆-alkyl cycloalkyl”, “C₁-C₆-alkyl heterocycloalkyl”.

“C₁-C₆-alkyl sulfonyloxy” refers to C₁-C₆-alkyl groups having a sulfonyloxy substituent, including 2-(methylsulfonyloxy)ethyl and the like.

“Sulfonyl” refers to group “—SO₂—R” wherein R is selected from H, “aryl”, “heteroaryl”, “C₁-C₆-alkyl”, “C₁-C₆-alkyl” substituted with halogens, e.g., an —SO₂—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynyl heteroaryl”, “C₁-C₆-alkyl cycloalkyl”, “C₁-C₆-alkyl heterocycloalkyl”.

“C₁-C₆-alkyl sulfonyl” refers to C₁-C₆-alkyl groups having a sulfonyl substituent, including 2-(methylsulfonyl)ethyl and the like.

“Sulfinyl” refers to a group “—S(O)—R” wherein R is selected from H, “C₁-C₆-alkyl”, “C₁-C₆-alkyl” substituted with halogens, e.g., an —SO—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynyl heteroaryl”, “C₁-C₆-alkyl cycloalkyl”, “C₁-C₆-alkyl heterocycloalkyl”.

“C₁-C₆-alkyl sulfinyl” refers to C₁-C₆-alkyl groups having a sulfinyl substituent, including 2-(methylsulfinyl)ethyl and the like.

“Sulfanyl” refers to groups —S—R where R includes H, “C₁-C₆-alkyl”, “C₁-C₆-alkyl” optionally substituted with halogens, e.g a —S—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynyl heteroaryl”, “C₁-C₆-alkyl cycloalkyl”, “C₁-C₆-alkyl heterocycloalkyl”. Preferred sulfanyl groups include methylsulfanyl, ethylsulfanyl, and the like.

“C₁-C₆-alkyl sulfanyl” refers to C₁-C₆-alkyl groups having a sulfanyl substituent, including 2-(ethylsulfanyl)ethyl and the like.

“Sulfonylamino” refers to a group —NRSO₂—R′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynyl heteroaryl”, “C₁-C₆-alkyl cycloalkyl”, “C₁-C₆-alkyl heterocycloalkyl”.

“C₁-C₆-alkyl sulfonylamino” refers to C₁-C₆-alkyl groups having a sulfonylamino substituent, including 2-(ethylsulfonylamino)ethyl and the like.

“Aminosulfonyl” refers to a group —SO₂—NRR′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynyl heteroaryl”, “C₁-C₆-alkyl cycloalkyl”, “C₁-C₆-alkyl heterocycloalkyl”.

“C₁-C₆-alkyl aminosulfonyl” refers to C₁-C₆-alkyl groups having an aminosulfonyl substituent, including 2-(cyclohexylaminosulfonyl)ethyl and the like.

“Substituted or unsubstituted”: Unless otherwise constrained by the definition of the indi-vidual substituent, the above set out groups, like “alkyl”, “alkenyl”, “alkynyl”, “aryl” and “heteroaryl” etc. groups can optionally be substituted with from 1 to 5 substituents selected from the group consisting of “C₁-C₆-alkyl”, “C₂-C₆-alkenyl”, “C₂-C₆-alkynyl”, “cycloalkyl”, “heterocycloalkyl”, “C₁-C₆-alkyl aryl”, “C₁-C₆-alkyl heteroaryl”, “C₁-C₆-alkyl cycloalkyl”, “C₁-C₆-alkyl heterocycloalkyl”, “amino”, “ammonium”, “acyl”, “acyloxy”, “acylamino”, “aminocarbonyl”, “alkoxycarbonyl”, “ureido”, “carbamate”, “aryl”, “heteroaryl”, “sulfinyl”, “sulfonyl”, “alkoxy”, “sulfanyl”, “halogen”, “carboxylic acid”, trihalomethyl, cyano, hydroxy, mercapto, nitro, and the like. Alternatively, said substitution could also comprise situations where neighbouring substituents have undergone ring closure, notably when vicinal functional substituents are involved, thus forming, e.g., lactams, lactons, cyclic anhydrides, but also acetals, thioacetals, aminals formed by ring closure for instance in an effort to obtain a protective group.

“Pharmaceutically acceptable salts or complexes” refers to salts or complexes of the below-specified compounds of Formula (I), (II), (III), (IV), (V), (VI) and (VII). Examples of such salts include, but are not restricted, to base addition salts formed by reaction of compounds of Formula (I), (II), (III), (IV), (V), (VI) and (VII) with organic or inorganic bases such as hydroxide, carbonate or bicarbonate of a metal cation such as those selected in the group consisting of alkali metals (sodium, potassium or lithium), alkaline earth metals (e.g. calcium or magnesium), ammonia, or with an organic primary, secondary or tertiary alkyl amine. Amine salts derived from methylamine, dimethylamine, trimethylamine, ethylamine, diethylamine, triethylamine, morpholine, N-Me-D-glucamine, N,N′-bis(phenylmethyl)-1,2-ethanediamine, tromethamine, ethanolamine, diethanolamine, ethylenediamine, N-methylmorpholine, procaine, piperidine, piperazine, arginine, choline, lysine and the like are contemplated being within the scope of the instant invention.

Also comprised are salts which are formed from to acid addition salts formed with inorganic acids (e.g. hydrochloric acid, hydrobromic acid, sulfuric acid, phosphoric acid, nitric acid, and the like), as well as salts formed with organic acids such as acetic acid, oxalic acid, tartric acid, citric acid, succinic acid, malic acid, fumaric acid, maleic acid, ascorbic acid, benzoic acid, tannic acid, palmitic acid, alginic acid, polyglutamic acid, naphthalene sulfonic acid, naphthalene disulfonic acid, and poly-galacturonic acid.

“Pharmaceutically active derivative” refers to any compound that upon administration to the recipient, is capable of providing directly or indirectly, the activity disclosed herein. The term “indirectly” also encompasses prodrugs which may be converted to the active form of the drug via endogenous enzymes or metabolism. Said prodrug is comprised of the active drug compound itself and a chemical masking group. Such masking group may be an ester moiety (e.g. obtained by masking a carboxylic acid or an hydroxy moiety of the compounds of Formula (I).

“Enantiomeric excess” (ee) refers to the products that are obtained by an asymmetric synthesis, i.e. a synthesis involving non-racemic starting materials and/or reagents or a synthesis comprising at least one enantioselective step, whereby a surplus of one enantiomer in the order of at least about 52% ee is yielded.

An “interferon” or “IFN”, as used herein, is intended to include any molecule defined as such in the literature, comprising for example any types of IFNs mentioned in the above section “Background of the Invention”. In particular, IFN-α, IFN-β and IFN-γ are included in the above definition. IFN-β is the preferred IFN according to the present invention. IFN-β suitable in accordance with the present invention is commercially available e.g. as Rebif® (Serono), Avonex® (Biogen) or Betaferon® (Schering).

The term “interferon-beta (IFN-beta or IFN-β)”, as used herein, is intended to include fibroblast interferon in particular of human origin, as obtained by isolation from biological fluids or as obtained by DNA recombinant techniques from prokaryotic or eukaryotic host cells, as well as its salts, functional derivatives, variants, analogs and active fragments. Preferably, IFN-beta is intended to mean recombinant Interferon beta-1a.

IFN-β suitable in accordance with the present invention is commercially available e.g. as Rebif® (Serono), Avonex® (Biogen) or Betaferon® (Schering). The use of interferons of human origin is also preferred in accordance with the present invention. The term interferon, as used herein, is intended to encompass salts, functional derivatives, variants, analogs and active fragments thereof.

The compounds used in the present invention also comprise their its tautomers, their geometrical isomers, their optically active forms as enantiomers, diastereoisomers and their racemate forms, as well as pharmaceutically acceptable salts and pharmaceutically active derivatives thereof. Preferred pharmaceutically acceptable salts of the compound used in the present invention are base addition salts formed by reaction of compounds with pharmaceutically acceptable bases like N-methyl-D-glucamine, tromethamine, lysine, arginine, choline, sodium, potassium or calcium salts of carbonates, bicarbonates or hydroxides.

The present invention relates to the use of Glepp-1 inhibitors for the treatment of an autoimmune and/or an inflammatory disorder alone or in combination with a further drug which is active in the treatment of autoimmune and/or an inflammatory disorders.

Any autoimmune and/or an inflammatory disorder is comprised by the present invention. Particular disorders comprised are: inflammatory bowel diseases, Crohn's disease, ulcerative colitis, collagenous colitis, lymphocytic colitis, diversion colitis irritable bowel syndrome, neuroinflammation including multiple sclerosis; Guillan Barre syndrome, chronic inflammatory polyneuropathy (CIPN), lung diseases including acute respiratory distress syndrome; joint and bone diseases including osteoarthritis and rheumatoid arthritis; liver diseases including liver fibrosis, cirrhosis and chronic liver disease; fibrotic diseases including, lupus, glomerulosclerosis, systemic sclerosis skin fibrosis, post-radiation fibrosis and cystic fibrosis; vascular pathologies including atherosclerosis, cardiomyopathy and myocardial infarction; restenosis; and degenerative diseases of the central nervous system including amyotrophic lateral sclerosis or inflammatory disorders of the skin including scleroderma and psoriasis.

In an embodiment, the compounds of the invention can be used in the treatment of inflammatory and/or autoimmune diseases, e.g. demyelinating diseases such as multiple sclerosis, alone or in combination with a co-agent useful in the treatment of autoimmune diseases.

A suitable co-agent for the treatment of a demyelinating disease is selected from the following compounds:
(a) Interferons, e.g. pegylated or non-pegylated interferons, e.g. administered by subcutaneous, intramuscular or oral routes, preferably interferon beta;
(b) Glatiramer, e.g. in the acetate form;
(c) Immunosuppressants with optionally antiproliferative/antineoplastic activity, e.g. mitoxantrone, methotrexate, azathioprine, cyclophosphamide, or steroids, e.g. methylprednisolone, prednisone or dexamethasone, or steroid-secreting agents, e.g. ACTH;
(d) Adenosine deaminase inhibitors, e.g. Cladribine;

Inhibitors of VCAM-1 expression or antagonists of its ligand, e.g. antagonists of the α4 μl integrin VLA-4 and/or alpha-4-beta-7 integrins, e.g. natalizumab (ANTEGREN). Rebif® (recombinant interferon-β) is the latest development in interferon therapy for multiple sclerosis (MS) and represents a significant advance in treatment. Rebif® is interferon (IFN)-beta 1a, produced from mammalian cell lines. It was established that interferon beta-1a given subcutaneously three times per week is efficacious in the treatment of Relapsing-Remitting Multiple Sclerosis (RRMS). Interferon beta-1a can have a positive effect on the long-term course of MS by reducing number and severity of relapses and reducing the burden of the disease and disease activity as measured by MRI. The dosing of IFN-beta in the treatment of relapsing-remitting MS according to the invention depends on the type of IFN-beta used.

Further anti-inflammatory agents (in particular for demyelinating diseases such as multiple sclerosis) are described below:

A further anti-inflammatory agent is Teriflunomide which is described in WO 02/080897

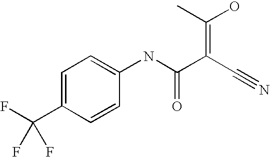

Still a further anti-inflammatory agent is Fingolimod which is described in EP-727,406 and WO 2004/028251.

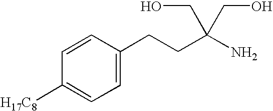

Still a further anti-inflammatory agent is Laquinimod which is described in WO 99/55678.

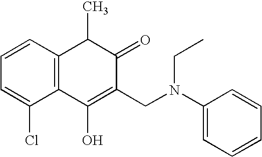

Still a further anti-inflammatory agent is Tensirolimus which is described in WO 02/28866.

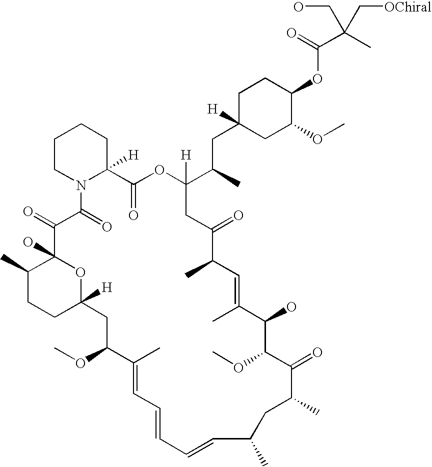

Still a further anti-inflammatory agent is Xaliprodene which is described in WO 98/48802.

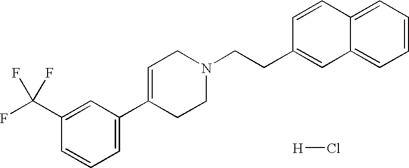

Still a further anti-inflammatory agent is Deskar Pirfenidone which is described in WO 03/068230.

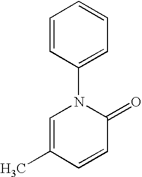

Still a further anti-inflammatory agent is the below benzothiazole derivative which is described in WO 01/47920.

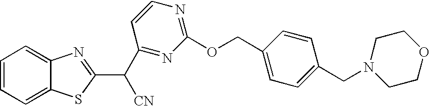

Still a further anti-inflammatory agent is the below hydroxamic acid derivative which is described in WO 03/070711.

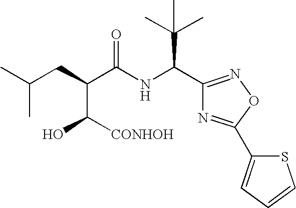

Still a further anti-inflammatory agent is MLN3897 which is described in WO2004/043965.

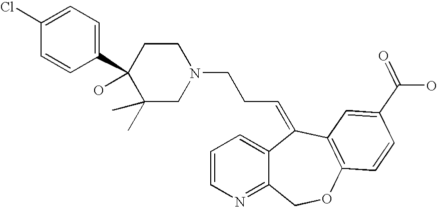

Still a further anti-inflammatory agent is CDP323 which is described in WO 99/67230.

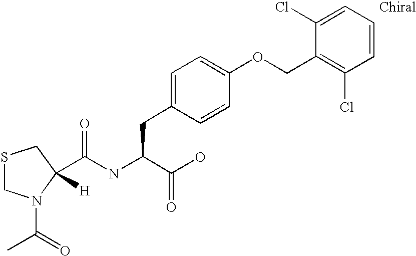

Still a further anti-inflammatory agent is Simvastatin which is described in WO 01/45698.

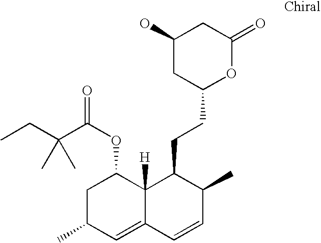

Still a further anti-inflammatory agent is Fampridine which is described in U.S. Pat. No. 5,540,938.

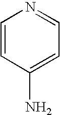

In a first embodiment the Glepp-1 inhibitor which may be used is a carboxylic acid of Formula (I):

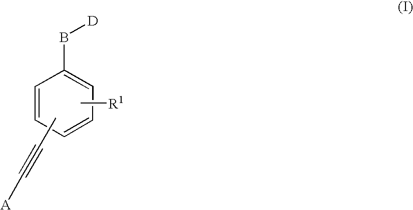

as well as its geometrical isomers, its optically active forms as enantiomers, diastereomers and its racemate forms, as well as pharmaceutically acceptable salts and pharmaceutically active derivatives thereof.

Compounds according to Formula (I) as well as their preparation are described in WO 2005/097773.

The substituents A, B, D and R¹ within Formula (I) are defined as follows:

A is selected from the group consisting of substituted or unsubstituted C₁-C₁₂-alkyl or C₁-C₈ alkyl, substituted or unsubstituted C₂-C₆-alkenyl, substituted or unsubstituted C₂-C₆-alkynyl, substituted or unsubstituted C₁-C₆-alkyl amine, substituted or unsubstituted C₁-C₆-alkyl alkoxy, substituted or unsubstituted aryl, substituted or unsubstituted heteroaryl, saturated or unsaturated substituted or unsubstituted C₃-C₈-cycloalkyl, substituted or unsubstituted heterocycloalkyl, substituted or unsubstituted C₁-C₆-alkyl aryl, substituted or unsubstituted C₁-C₆-alkyl heteroaryl, substituted or unsubstituted C₂-C₆-alkenyl aryl, substituted or unsubstituted C₂-C₆-alkenyl heteroaryl, substituted or unsubstituted C₂-C₆-alkynyl aryl, substituted or unsubstituted C₂-C₆-alkynyl heteroaryl, substituted or unsubstituted C₁-C₆-alkyl cycloalkyl, substituted or unsubstituted C₁-C₆-alkyl heterocycloalkyl, substituted or unsubstituted C₂-C₆-alkenyl cycloalkyl, substituted or unsubstituted C₂-C₆-alkenyl heterocycloalkyl, substituted or unsubstituted C₂-C₆-alkynyl cycloalkyl, substituted or unsubstituted C₂-C₆-alkynyl heterocycloalkyl. In a preferred embodiment A is C₄-C₆ alkyl aryl, and in particular n-butylphenyl.

R¹ is selected from the group consisting of H, substituted or unsubstituted C₁-C₆-alkyl, substituted or unsubstituted C₁-C₆-alkoxy, halogen. In a specific embodiment R¹ is H.

B is either an amine selected from the group consisting of:

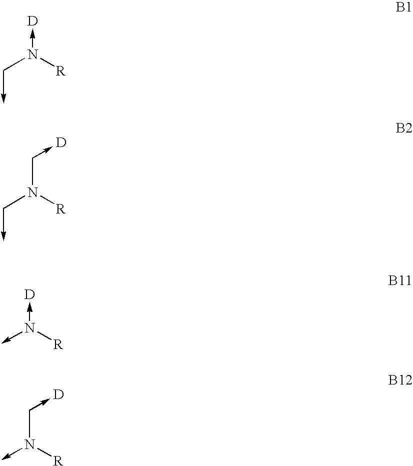

or an ether of the formula:

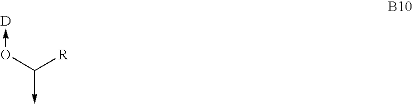

or a carboxamide selected from the group consisting of:

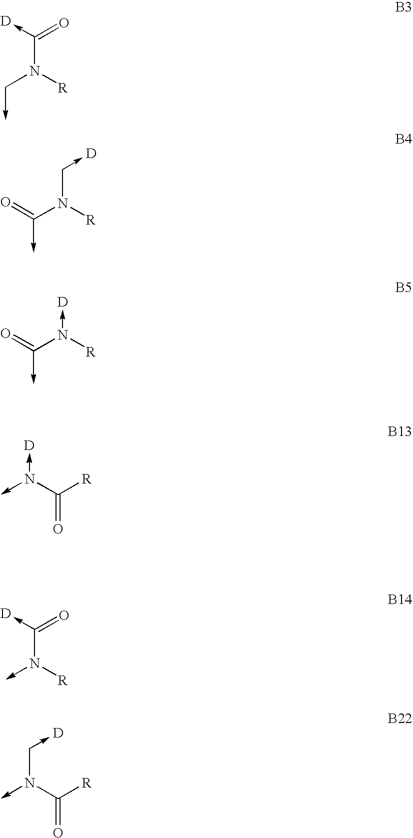

or a sulfonamide selected from the group consisting of:

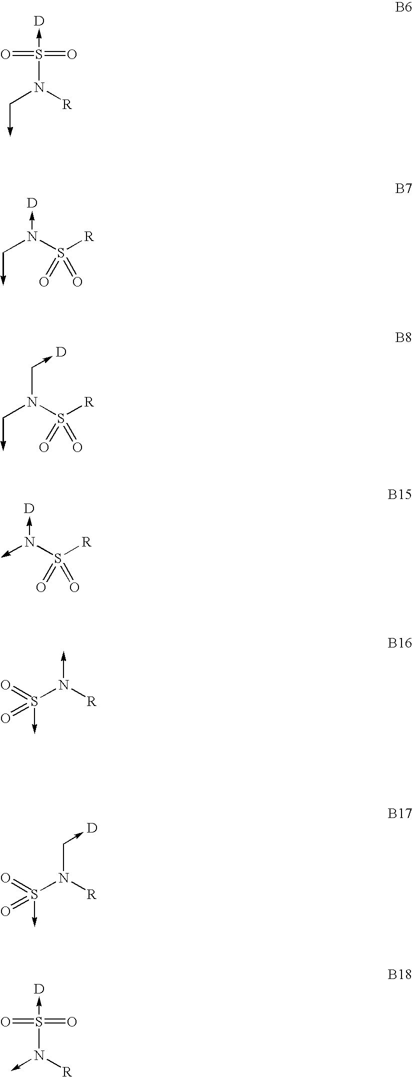

or a urea moiety selected from the group consisting of:

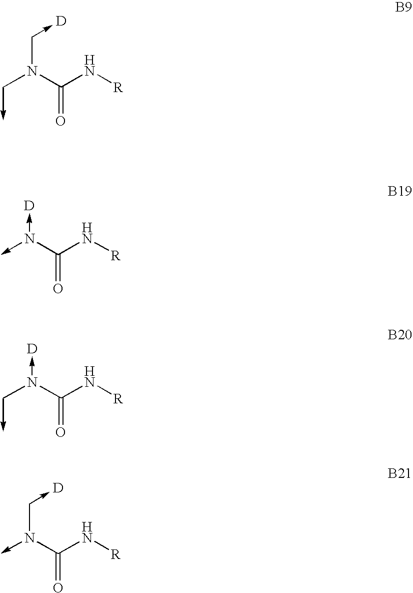

wherein D is either selected from the group consisting of D1, D2, D3 herebelow

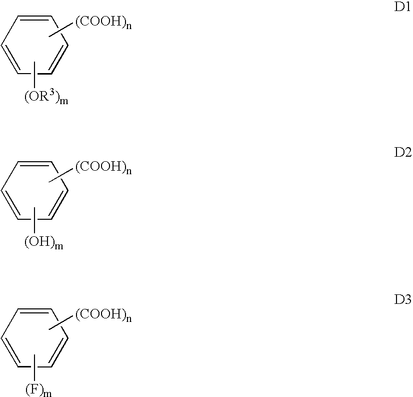

with m being an integer selected from 0, 1 or 2 and n being an integer selected from 1 or 2 and R³ is H or C₁-C₆ alkyl; or D4

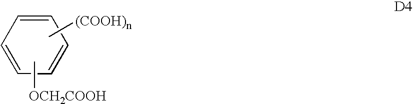

with n being an integer selected from 0 or 1, and

wherein R is selected from the group consisting of substituted or unsubstituted C₁-C₁₂-alkyl or C₁-C₈ alkyl, substituted or unsubstituted C₂-C₆-alkenyl, substituted or unsubstituted C₂-C₆-alkynyl, substituted or unsubstituted C₁-C₆-alkoxy (including ethers or polyethers), substituted or unsubstituted C₁-C₆-alkyl amine, substituted or unsubstituted C₁-C₆-alkyl alkoxy, substituted or unsubstituted aryl, substituted or unsubstituted heteroaryl, saturated or unsaturated substituted or unsubstituted C₃-C₈-cycloalkyl, substituted or unsubstituted heterocycloalkyl, substituted or unsubstituted C₁-C₆-alkyl aryl (e.g. a benzyl group), substituted or unsubstituted C₁-C₆-alkyl heteroaryl, substituted or unsubstituted C₂-C₆-alkenyl aryl, substituted or unsubstituted C₂-C₆-alkenyl heteroaryl, substituted or unsubstituted C₂-C₆-alkynyl aryl, substituted or unsubstituted C₂-C₆-alkynyl heteroaryl, substituted or unsubstituted C₁-C₆-alkyl cycloalkyl, substituted or unsubstituted C₁-C₆-alkyl heterocycloalkyl, substituted or unsubstituted C₂-C₆-alkenyl cycloalkyl, substituted or unsubstituted C₂-C₆-alkenyl heterocycloalkyl, substituted or unsubstituted C₂-C₆-alkynyl cycloalkyl, substituted or unsubstituted C₂-C₆-alkynyl heterocycloalkyl.

Aryl or heteroaryl moities in R and A include phenyl, naphthyl, phenanthrenyl, pyrrolyl, furyl, thienyl, imidazolyl, pyridyl, oxazolyl, isoxazolyl, thiazolyl, isothiazolyl, pyrazolyl, 1,2,3-triazolyl, 1,2,4-triazolyl, 1,2,3-oxadiazolyl, benzo(1,2,5)oxadiazolyl, 1,2,4-oxadiazolyl, 1,2,5-oxadiazolyl, 1,3,4-oxadiazolyl, tetrazolyl, 1,3,4-triazinyl, 1,2,3-triazinyl, benzopyrimidinyl, benzodioxolyl, benzofuryl, [2,3-dihydro]benzofuryl, isobenzofuryl, benzothienyl, indazolyl, benzotriazolyl, isobenzothienyl, indolyl, isoindolyl, 3H-indolyl, benzimidazolyl, benzothiazolyl, benzoxazolyl, pyridazinyl, pyrimidyl, quinolizinyl, quinazolinyl, phthalazinyl, quinoxalinyl, cinnolinyl, naphthyridinyl, quinolyl, isoquinolyl, tetrazolyl, 5,6,7,8-tetrahydroquinolyl, 5,6,7,8-tetrahydroisoquinolyl, purinyl, pteridinyl, xanthenyl, benzoquinolyl, oxolanyl, pyrrolidinyl, pyrazolidinyl, 2H-benzo[d]1,3-dioxolenyl, indanyl, imidazolidinyl, 1,2,4-oxadiazolidinyl, 1,2,5-oxadiazolidinyl, 1,3,4-oxadiazolidinyl or isoxazolidinyl.

In a specific embodiment the aryl or heteroaryl moieties are: phenyl, pyridyl, pyrazolyl, benzodioxolyl, benzofuryl, benzothienyl, indazolyl.

Cycloalkyl moities in R and A include in particular cyclopentyl, or cyclohexyl groups.

A further specific embodiment consists in carboxylic acid of Formula (I), wherein B is either of

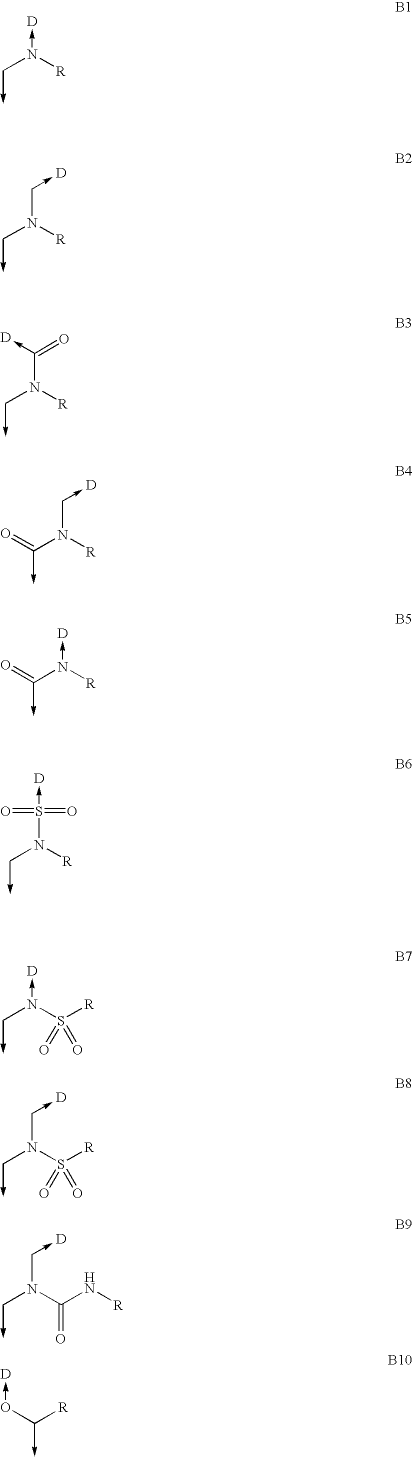

and in a preferred embodiment B is either B1 or B2 or B3.

In another specific embodiment according to Formula (I) B is either of B12, B16, B17, B20 or B22.

A further specific embodiment consists in carboxylic acid of Formula (I), wherein D is either of:

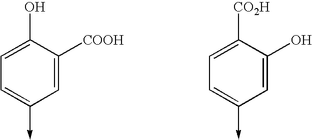

In a further specific embodiment D is

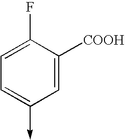

In still a further specific embodiment R is a C₄-C₆-alkyl, e.g. a hexyl group.

Preferred compounds of the invention are those of Formula (I), wherein A is a phenyl group substituted by a C₁-C₄-alkyl or a halogen; B is either B1, B2, B3, B12; R is a substituted or unsubstituted C₄-C₆-alkyl, a substituted or unsubstituted C₃-C₈-cycloalkyl, a substituted or unsubstituted C₁-C₆-alkyl cycloalkyl, e.g. a methyl substituted by C₃-C₈-cycloalkyl (like a cyclopentyl or a cyclopropyl moiety); and D is

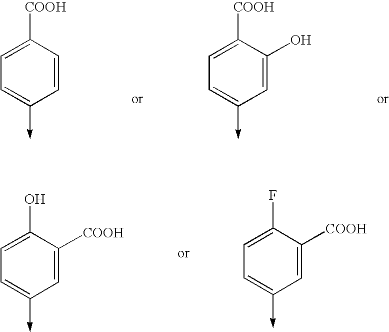

A further specific embodiment is related to compounds of Formula (Ia) wherein the substituent ethynyl-A is in the para-position as set out below:

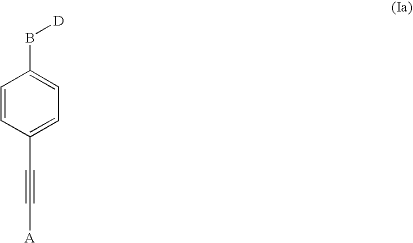

A specific embodiment consists in carboxylic acid of Formula (Ia) wherein A is an aryl moiety, in particular a substituted or unsubstituted phenyl group. A specific phenyl would be a phenyl being substituted by a C₁-C₈-alkyl, more preferably by a C₁-C₄-alkyl, a halogen or an alkoxy group, e.g. a butyl, trifluoromethyl group or a chlorine.

A further specific embodiment is related to compounds of Formula (Ib) and (Ic) wherein the substituent ethynyl-A is in the para-position or orto-positon as set out below

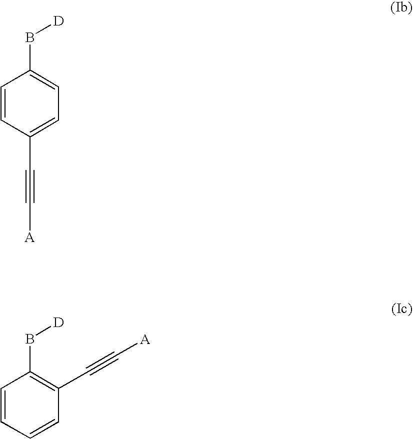

wherein A is a C₁-C₆-alkyl aryl, thiazolyl; B is B1, B3, B4, B5, B11, B14, B18; R and D are as defined above.

Specific Glepp-1 inhibitors according to Formula (I) are selected from the group of:
5-[{4-[(4-butylphenyl)ethynyl]benzyl}(hexyl)amino]-2-hydroxybenzoic acid
5-[{4-[(4-butylphenyl)ethynyl]benzyl}(hexyl)amino]-2-fluorobenzoic acid
4-({{4-[(4-butylphenyl)ethynyl]benzyl}[2-(4-chlorophenyl)ethyl]amino}methyl)benzoic acid
5-[{4-[(4-butylphenyl)ethynyl]benzyl}(3-phenylpropyl)amino]-2-hydroxybenzoic acid
5-[{4-[(4-butylphenyl)ethynyl]benzyl}(1-naphthylmethyl)amino]-2-hydroxybenzoic acid
5-((4-tert-butylbenzyl){4-[(4-butylphenyl)ethynyl]benzyl}amino)-2-hydroxybenzoic acid
4-[{4-[(4-butylphenyl)ethynyl]benzyl}(hexyl)amino]-2-hydroxybenzoic acid
2-fluoro-5-{hexyl[4-(phenylethynyl)benzyl]amino}benzoic acid
5-[{4-[(4-chlorophenyl)ethynyl]benzyl}(hexyl)amino]-2-hydroxybenzoic acid
5-(hexyl {4-[(4-methoxyphenyl)ethynyl]benzyl}amino)-2-hydroxybenzoic acid
5-[hexyl(4-{[4-(trifluoromethyl)phenyl]ethynyl}benzyl)amino]-2-hydroxybenzoic acid
5-[{4-[(4-butylphenyl)ethynyl]benzyl}(cyclopentylmethyl)amino]-2-fluorobenzoic acid
5-[{4-[(4-butylphenyl)ethynyl]benzyl}(3,3-dimethylbutyl)amino]-2-fluorobenzoic acid
5-((cyclopentylmethyl){4-[(4-methoxyphenyl)ethynyl]benzyl}amino)-2-hydroxybenzoic acid
5-[{4-[(4-butylphenyl)ethynyl]benzyl}(ethyl)amino]-2-fluorobenzoic acid
5-(hexyl {4-[(4-propylphenyl)ethynyl]benzyl}amino)-2-hydroxybenzoic acid
5-[{4-[(4-butylphenyl)ethynyl]benzyl}(pentyl)amino]-2-fluorobenzoic acid
5-[{4-[(4-butylphenyl)ethynyl]benzyl}(methyl)amino]-2-fluorobenzoic acid
5-[{4-[(4-butylphenyl)ethynyl]benzyl}(cyclopropylmethyl)amino]-2-fluorobenzoic acid
5-{butyl[4-(phenylethynyl)benzyl]amino}-2-fluorobenzoic acid
2-fluoro-5-[[4-(phenylethynyl)benzyl](propyl)amino]benzoic acid
2-fluoro-5-[{4-[(4-fluorophenyl)ethynyl]benzyl}(hexyl)amino]benzoic acid
2-fluoro-5-(hexyl {4-[(4-propylphenyl)ethynyl]benzyl}amino)benzoic acid
5-{{4-[(4-butylphenyl)ethynyl]benzyl}[(2-carboxycyclopropyl)methyl]amino}-2-fluorobenzoic acid
5-[{4-[(4-ethylphenyl)ethynyl]benzyl}(hexyl)amino]-2-fluorobenzoic acid
5-[{4-[(4-tert-butylphenyl)ethynyl]benzyl}(hexyl)amino]-2-fluorobenzoic acid
5-{[{4-[(4-butylphenyl)ethynyl]phenyl}(hexyl)amino]methyl}-2-fluorobenzoic acid
5-[{4-[(4-butylphenyl)ethynyl]benzyl}(isobutyl)amino]-2-fluorobenzoic acid
5-[(1-{4-[(4-butylphenyl)ethynyl]phenyl}pentyl)oxy]-2-hydroxybenzoic acid
5-{[{4-[(4-butylphenyl)ethynyl]benzyl}(hexyl)amino]carbonyl}-2-hydroxybenzoic acid
5-{[{4-[(4-butylphenyl)ethynyl]benzoyl}(hexyl)amino]methyl}-2-hydroxybenzoic acid
5-{[{2-[(4-butylphenyl)ethynyl]benzyl}(hexyl)amino]carbonyl}-2-hydroxybenzoic acid
4-((3-cyclopentylpropyl){4-[(4-fluorophenyl)ethynyl]benzoyl}amino)-2-hydroxybenzoic acid
4-[{4-[(4-butylphenyl)ethynyl]benzoyl}(3-cyclopentylpropyl)amino]-2-hydroxybenzoic acid
5-{[{4-[(4-fluorophenyl)ethynyl]benzoyl}(hexyl)amino]methyl}-2-hydroxybenzoic acid
5-{[{4-[(4-chlorophenyl)ethynyl]benzoyl}(hexyl)amino]methyl}-2-hydroxybenzoic acid
5-{[{4-[(4-butylphenyl)ethynyl]benzyl}(hexyl)amino]carbonyl}-2-fluorobenzoic acid
5-[{4-[(4-butylphenyl)ethynyl]benzoyl}(hexyl)amino]-2-fluorobenzoic acid
5-{[{4-[(4-butylphenyl)ethynyl]benzoyl}(hexyl)amino]methyl}-2-fluorobenzoic acid
4-[{4-[(4-chlorophenyl)ethynyl]benzoyl}(3-cyclopentylpropyl)amino]-2-hydroxybenzoic acid

In a further embodiment the Glepp-1 inhibitor is an alkynyl aryl carboxamide according to Formula (II), which Formula (II) is selected from any of Formulae (IIa), (IIb), (IIc) or (IId):

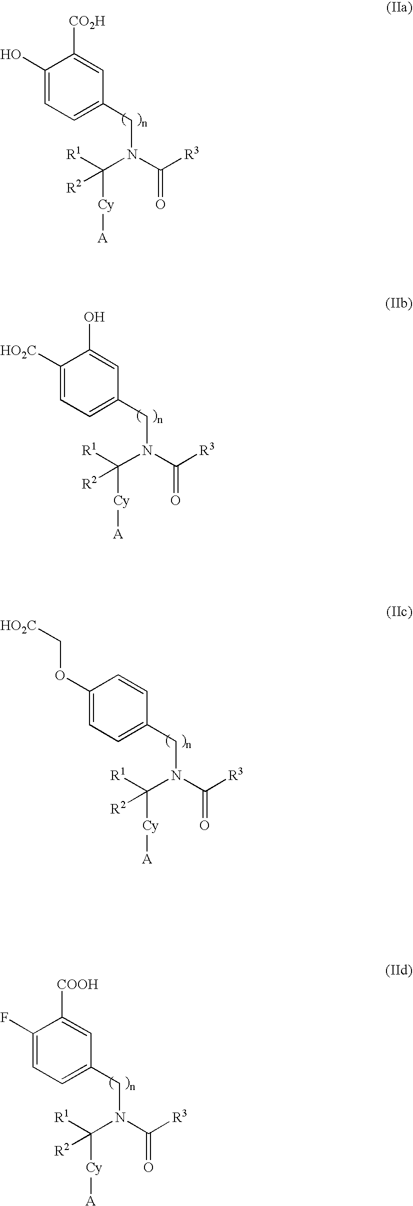

as well as its geometrical isomers, its optically active forms as enantiomers, diastereomers and its racemate forms, as well as pharmaceutically acceptable salts and pharmaceutically active derivatives thereof.

Compounds according to Formula (IIa), (IIb), (IIc) and (IId) as well as their preparation are described in WO 2005/012280.

In said Formulae:

A is a substituted or unsubstituted C₂-C₁₅ alkynyl, substituted or unsubstituted C₂-C₆-alkynyl aryl, substituted or unsubstituted C₂-C₆-alkynyl heteroaryl.

Cy is a substituted or unsubstituted aryl, substituted or unsubstituted heteroaryl, substituted or unsubstituted C₃-C₈-cycloalkyl or substituted or unsubstituted heterocycle group

Said aryl or heteroaryl moities in Cy include phenyl, naphthyl, phenanthrenyl, pyrrolyl, furyl, thienyl, imidazolyl, pyridyl, oxazolyl, isoxazolyl, thiazolyl, isothiazolyl, pyrazolyl, 1,2,3-triazolyl, 1,2,4-triazolyl, 1,2,3-oxadiazolyl, benzo(1,2,5)oxadiazolyl, 1,2,4-oxadiazolyl, 1,2,5-oxadiazolyl, 1,3,4-oxadiazolyl, tetrazolyl, 1,3,4-triazinyl, 1,2,3-triazinyl, benzo-pyrimidinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, benzothiazolyl, benzoxazolyl, pyridazinyl, pyrimidyl, quinolizinyl, quinazolinyl, pthalazinyl, quinoxalinyl, cinnolinyl, napthyridinyl, quinolyl, isoquinolyl, tetrazolyl, 5,6,7,8-tetrahydroquinolyl, 5,6,7,8-tetrahydroisoquinolyl, purinyl, pteridinyl, xanthenyl, benzoquinolyl, oxolanyl, pyrrolidinyl, pyrazolidinyl, 2H-benzo[d]1,3-dioxolenyl, indanyl, imidazolidinyl, 1,2,4-oxadiazolidinyl, 1,2,5-oxadiazolidinyl, 1,3,4-oxadiazolidinyl or isoxazolidinyl.

According to one embodiment, Cy is a substituted or unsubstituted phenyl. Also comprised are diaryl (e.g. biphenyl), or di-heteroaryl, or aryl-heteroaryl (e.g. phenyl-thiazolyl, or heteroaryl-aryl (e.g. thiazolyl-phenyl) moieties.

n is 0 or 1.

R¹ and R² are each independently selected from the group consisting of hydrogen or substituted or unsubstituted C₁-C₆-alkyl. According to one embodiment both R¹ and R² are hydrogen.

R³ is selected from the group consisting of substituted or unsubstituted C₁-C₆-alkyl, substituted or unsubstituted C₂-C₆-alkenyl, substituted or unsubstituted C₂-C₆-alkynyl, substituted or unsubstituted C₁-C₆-alkoxy, substituted or unsubstituted C₁-C₆-alkyl amine, substituted or unsubstituted C₁-C₆-alkyl alkoxy, substituted or unsubstituted C₁-C₆-alkyl carboxy, substituted or unsubstituted aryl, substituted or unsubstituted heteroaryl, substituted or unsubstituted saturated or unsaturated C₃-C₈-cycloalkyl, substituted or unsubstituted heterocycloalkyl, acyl moiety, substituted or unsubstituted C₁-C₆-alkyl aryl, substituted or unsubstituted C₁-C₆-alkyl heteroaryl, substituted or unsubstituted C₂-C₆-alkenyl aryl, substituted or unsubstituted C₂-C₆-alkenyl heteroaryl, substituted or unsubstituted C₂-C₆-alkynyl aryl, substituted or unsubstituted C₂-C₆-alkynyl heteroaryl, substituted or unsubstituted C₁-C₆-alkyl cycloalkyl, substituted or unsubstituted C₁-C₆-alkyl heterocycloalkyl, substituted or unsubstituted C₂-C₆-alkenyl cycloalkyl, substituted or unsubstituted C₂-C₆-alkenyl heterocycloalkyl, substituted or unsubstituted C₂-C₆-alkynyl cycloalkyl, substituted or unsubstituted C₂-C₆-alkynyl heterocycloalkyl.

In a particular embodiment R¹ and R² are each H, Cy is a phenyl group, A is a moiety of the formula —C≡C—R⁶ wherein R⁶ is phenyl, C₁-C₁₂ alkyl phenyl, C₂-C₆-alkenyl phenyl, C₂-C₆-alkynyl phenyl.

Specific Glepp-1-inhibitors are selected from the group consisting of:
4-[{4-[(4-butylphenyl)ethynyl]benzyl}(3-cyclopentylpropanoyl)amino]-2-hydroxy-benzoic acid
5-[{4-[(4-butylphenyl)ethynyl]benzyl}(cyclohexylcarbonyl)amino]-2-hydroxybenzoic acid
5-[{4-[(4-butylphenyl)ethynyl]benzyl}(hexanoyl)amino]-2-hydroxybenzoic acid
5-((4-tert-butylbenzoyl){4-[(4-butylphenyl)ethynyl]benzyl}amino)-2-hydroxybenzoic acid
5-((biphenyl-4-ylcarbonyl){4-[(4-butylphenyl)ethynyl]benzyl}amino)-2-hydroxybenzoic acid
5-[{4-[(4-butylphenyl)ethynyl]benzyl}(3,3-dimethylbutanoyl)amino]-2-hydroxybenzoic acid
5-((1,3-benzodioxol-5-ylcarbonyl){4-[(4-butylphenyl)ethynyl]benzyl}amino)-2-hydroxybenzoic acid
5-([(benzyloxy)acetyl]{4-[(4-butylphenyl)ethynyl]benzyl}amino)-2-hydroxybenzoic acid
5-[{4-[(4-butylphenyl)ethynyl]benzyl}(4-hexylbenzoyl)amino]-2-hydroxybenzoic acid
5-((1-benzothien-2-ylcarbonyl){4-[(4-butylphenyl)ethynyl]benzyl}amino)-2-hydroxybenzoic acid
5-{[{4-[(4-butylphenyl)ethynyl]benzyl}(hexanoyl)amino]methyl}-2-hydroxybenzoic acid
(4-{[{4-[(4-butylphenyl)ethynyl]benzyl}(hexanoyl)amino]methyl}phenoxy)acetic acid
8-[{4-[(4-butylphenyl)ethynyl]benzyl}(3-cyclopentylpropanoyl)amino]-5,6,7,8-tetrahydronaphthalene-2-carboxylic acid
4-{[{4-[(4-butylphenyl)ethynyl]benzyl}(3-cyclopentylpropanoyl)amino]methyl}-benzoic acid
5-[{4-[(4-chlorophenyl)ethynyl]benzyl}(2-thienylacetyl)amino]-2-hydroxybenzoic acid
5-[{4-[(4-chlorophenyl)ethynyl]benzyl}(2-thienylacetyl)amino]-2-hydroxybenzoic acid
5-[{4-[(4-butylphenyl)ethynyl]benzyl}(3-cyclopentylpropanoyl)amino]-2-fluorobenzoic acid
5-[{4-[(4-butylphenyl)ethynyl]benzyl}(3,3-dimethylbutanoyl)amino]-2-fluorobenzoic acid
4-[{4-[(4-butylphenyl)ethynyl]benzyl}(cyclohexylcarbonyl)amino]-2-hydroxybenzoic acid
4-[{4-[(4-butylphenyl)ethynyl]benzyl}(hexanoyl)amino]-2-hydroxybenzoic acid
4-[{4-[(4-butylphenyl)ethynyl]benzyl}(3-cyclopentylpropanoyl)amino]-2-fluorobenzoic acid
4-[{4-[(4-tert-butylphenyl)ethynyl]benzyl}(3-cyclopentylpropanoyl)amino]-2-hydroxybenzoic acid
4-((3-cyclopentylpropanoyl){4-[(4-propoxyphenyl)ethynyl]benzyl}amino)-2-hydroxybenzoic acid
4-((3-cyclopentylpropanoyl){4-[(4-propylphenyl)ethynyl]benzyl}amino)-2-hydroxybenzoic acid
5-{[{4-[(4-butylphenyl)ethynyl]benzyl}(3-cyclopentylpropanoyl)amino]methyl}-2-hydroxybenzoic acid

In a further embodiment the Glepp-1 inhibitor is an aryl dicarboxamide according to Formula (III), which Formula (III) is selected from any of the Formulae (IIIa), (IIIb) or (IIIc):

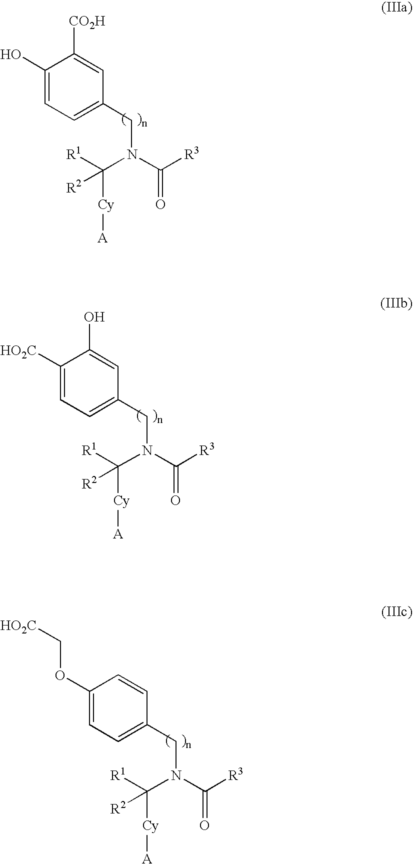

as well as its geometrical isomers, its optically active forms as enantiomers, diastereomers and its racemate forms, as well as pharmaceutically acceptable salts and pharmaceutically active derivatives thereof.

Compounds according to Formulae (IIIa), (IIIb) and (IIIc) as well as their preparation are described in WO 2005/011685.

In said Formulae:

A is an aminocarbonyl moiety of the formula —CO—NHR⁶ wherein R⁶ is C₆-C₁₅ alkyl, C₂-C₁₅-alkenyl, C₂-C₁₅-alkynyl, a C₃-C₈-cycloalkyl, C₁-C₆ alkyl-C₃-C₈-cycloalkyl, phenyl, C₁-C₁₂ alkyl phenyl, C₂-C₆-alkenyl phenyl, C₂-C₆-alkynyl phenyl.

Cy is a substituted or unsubstituted aryl, substituted or unsubstituted heteroaryl, substituted or unsubstituted aryl-heteroaryl, substituted or unsubstituted heteroaryl-aryl, substituted or unsubstituted aryl-aryl, substituted or unsubstituted cycloalkyl or substituted or unsubstituted heterocycle group.

Such aryl or heteroaryl include phenyl, naphthyl, phenanthrenyl, pyrrolyl, furyl, thienyl, imidazolyl, pyridyl, oxazolyl, isoxazolyl, thiazolyl, isothiazolyl, pyrazolyl, 1,2,3-triazolyl, 1,2,4-triazolyl, 1,2,3-oxadiazolyl, benzo(1,2,5)oxadiazolyl, 1,2,4-oxadiazolyl, 1,2,5-oxadiazolyl, 1,3,4-oxadiazolyl, tetrazolyl, 1,3,4-triazinyl, 1,2,3-triazinyl, benzopyrimidinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, benzothiazolyl, benzoxazolyl, pyridazinyl, pyrimidyl, quinolizinyl, quinazolinyl, pthalazinyl, quinoxalinyl, cinnolinyl, napthyridinyl, quinolyl, isoquinolyl, tetrazolyl, 5,6,7,8-tetrahydroquinolyl, 5,6,7,8-tetrahydroisoquinolyl, purinyl, pteridinyl, xanthenyl, benzoquinolyl, oxolanyl, pyrrolidinyl, pyrazolidinyl, 2H-benzo[d]1,3-dioxolenyl, indanyl, imidazolidinyl, 1,2,4-oxadiazolidinyl, 1,2,5-oxadiazolidinyl, 1,3,4-oxadiazolidinyl or isoxazolidinyl.

According to one embodiment Cy is a substituted or unsubstituted phenyl, substituted or unsubstituted thiazolyl, substituted or unsubstituted phenyl-thiazolyl, substituted or unsubstituted thiazolyl-phenyl.

n is either 0 or 1.

R¹ and R² are each independently selected from the group consisting of hydrogen or substituted or unsubstituted C₁-C₆-alkyl. According to one embodiment both R¹ and R² are hydrogen.

R³ is selected from the group consisting of substituted or unsubstituted C₁-C₆-alkyl, substituted or unsubstituted C₂-C₆-alkenyl, substituted or unsubstituted C₂-C₆-alkynyl, substituted or unsubstituted C₁-C₆-alkoxy, substituted or unsubstituted C₁-C₆-alkyl amine, substituted or unsubstituted C₁-C₆-alkyl alkoxy, substituted or unsubstituted aryl, substituted or unsubstituted heteroaryl, substituted or unsubstituted saturated or unsaturated C₃-C₈-cycloalkyl, substituted or unsubstituted heterocycloalkyl, acyl moiety, substituted or unsubstituted C₁-C₆-alkyl aryl, substituted or unsubstituted C₁-C₆-alkyl heteroaryl, substituted or unsubstituted C₂-C₆-alkenyl aryl, substituted or unsubstituted C₂-C₆-alkenyl heteroaryl, substituted or unsubstituted C₂-C₆-alkynyl aryl, substituted or unsubstituted C₂-C₆-alkynyl heteroaryl, substituted or unsubstituted C₁-C₆-alkyl cycloalkyl, substituted or unsubstituted C₁-C₆-alkyl heterocycloalkyl, substituted or unsubstituted C₂-C₆-alkenyl cycloalkyl, substituted or unsubstituted C₂-C₆-alkenyl heterocycloalkyl, substituted or unsubstituted C₂-C₆-alkynyl cycloalkyl, substituted or unsubstituted C₂-C₆-alkynyl heterocycloalkyl.

In a specific embodiment R¹ and R² are each H, Cy is selected from the group consisting of phenyl, thiazolyl, phenyl-thiazolyl, thiazolyl-phenyl and R⁶ is selected from the group consisting of C₈-C₁₂-alkyl, C₁-C₄-alkyl phenyl which may be substituted by C₁-C₈-alkyl or phenoxy.

Specific Glepp-1 inhibitors are selected from the group consisting of:
5-[(3-cyclopentylpropanoyl)(4-{[(4-phenoxybenzyl)amino]carbonyl}-benzyl)amino]-2-hydroxybenzoic acid
5-((4-cyanobenzoyl){[2-(4-{[(4-pentylbenzyl)amino]carbonyl}phenyl)-1,3-thiazol-4-yl]methyl}amino)-2-hydroxybenzoic acid
(4-{[{[2-(4-{[(4-pentylbenzyl)amino]carbonyl}phenyl)-1,3-thiazol-4-yl]methyl}(3-phenylpropanoyl)amino]methyl}phenoxy)acetic acid
(4-{[[(2-{4-[(octylamino)carbonyl]phenyl}-1,3-thiazol-4-yl)methyl](3-phenylpropanoyl)-amino]methyl}phenoxy)acetic acid

In still a further embodiment the Glepp-1 inhibitor is a substituted methylene amide derivative of Formula (IV):

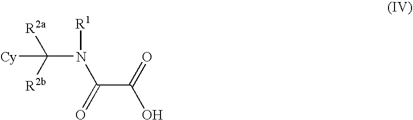

as well as its geometrical isomers, its optically active forms as enantiomers, diastereomers and its racemate forms, as well as pharmaceutically acceptable salts and pharmaceutically active derivatives thereof.

Compounds according to Formula (IV) as well as their preparation is described in WO 03/064376.

In said Formula:

R¹ is selected from the group consisting of substituted or unsubstituted C₁-C₁₂-alkyl, substituted or unsubstituted C2-C12-alkenyl, substituted or unsubstituted C₁-C₁₂-alkynyl, substituted or unsubstituted aryl, substituted or unsubstituted heteroaryl, substituted or unsubstituted C₃-C₈-cycloalkyl or hetero-cycloalkyl, substituted or unsubstituted C₁-C₁₂-alkyl-aryl or substituted or unsubstituted C₁-C₁₂-alkyl-heteroaryl, substituted or unsubstituted C₂-C₁₂-alkenyl-aryl or -heteroaryl, substituted or unsubstituted C₂-C₁₂-alkynyl-aryl or -heteroaryl.

R²¹ and R^{2b} are each independently selected from the group comprising or consisting of H or substituted or unsubstituted C₁-C₁₂-alkyl.

Cy is a substituted or unsubstituted aryl, substituted or unsubstituted heteroaryl, substituted or unsubstituted cycloalkyl or substituted or unsubstituted heterocycle group.

In a specific embodiment R^{2a} and R^{2b} are each H; R¹ is —CA₁A₂A₃ with A₁, A₂, A₃ being hydrogen, methyl, ethyl, propyl, phenyl or thienyl, optionally substituted by cyano, halogen, methoxy, hydroxy, phenoxy, —NO₂, trifluoromethyl; and Cy is a thienyl, phenyl or biphenyl being substituted by —SO₂R³, —CO—NR³R^{3′} in which R^{3′} is H and R³ is C₇-C₁₅-alkyl, particularly C₈-C₁₅-alkyl and more particularly a dodecyl group which may be substituted by a halogen, with the proviso that at least two of A₁, A₂, A₃ is not hydrogen or methyl, ethyl, propyl. In a further specific embodiment the Glepp-1 inhibitor has the Formula (V):

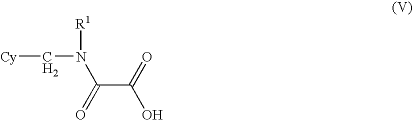

as well as its geometrical isomers, its optically active forms as enantiomers, diastereomers and its racemate forms, as well as pharmaceutically acceptable salts and pharmaceutically active derivatives thereof.

Compounds according to Formula (V) as well as their preparation is described in WO 03/064376.

In said Formula:

R¹ is selected from the group consisting of phenyl, benzyl, phenethyl, 1-methylbenzyl which may be substituted by C₁-C₆-alkyl group or a cycloalkyl group;

Cy is a phenyl or a biphenyl group substituted with a moiety selected from the group consisting of —NH—CO—R³, —CO—NH—R³, or an oxadiazole group substituted with R³, wherein R³ is C₇-C₁₅-alkyl, particularly C₈-C₁₅-alkyl and more particularly a dodecyl group.

Specific Glepp-inhibitors according to Formula (IV) and (V) are selected from the group consisting of:
{{4-[(4-hexylphenyl)ethynyl]benzyl}[4-(trifluoromethyl)benzyl]amino}-(oxo)acetic acid
(benzyl {4-[(pentadecylamino)carbonyl]benzyl}amino)(oxo)acetic acid
({[1-(tert-butoxycarbonyl)-4-piperidinyl]methyl}{4-[(dodecylamino)carbonyl]benzyl}-amino)(oxo)acetic acid
oxo {{4-[(9E)-9-tetradecenoylamino]benzyl}[4-(trifluoromethyl)benzyl]amino}acetic acid,
oxo {[4-(trifluoromethyl)benzyl][4-(3-undecyl-1,2,4-oxadiazol-5-yl)benzyl]amino}acetic acid
oxo {(4-tetradec-1-ynylbenzyl)[4-(trifluoromethyl)benzyl]amino}acetic acid
{(4-dodec-1-ynylbenzyl)[4-(trifluoromethyl)benzyl]amino}(oxo)acetic acid
oxo{{1-[4-(trifluoromethyl)phenyl]ethyl}[4-(3-undecyl-1,2,4-oxadiazol-5-yl)benzyl]-amino}acetic acid
([(2-butyl-1-benzofuran-3-yl)methyl]{4-[(dodecylamino)carbonyl]benzyl}amino)-(oxo)acetic acid
{(4-{[(4-octylphenyl)amino]carbonyl}benzyl)[4-(trifluoromethyl)benzyl]amino}(oxo)-acetic acid
{{cyclopentyl[4-(trifluoromethyl)phenyl]methyl}[4-(tridecanoylamino)benzyl]amino}-(oxo)acetic acid
oxo([4-(trifluoromethyl)benzyl]{[4-(3-undecyl-1,2,4-oxadiazol-5-yl)-1-naphthyl]methyl}-amino)acetic acid
{{cyclopentyl[4-(trifluoromethyl)phenyl]methyl}[4-(3-undecyl-1,2,4-oxadiazol-5-yl)benzyl]amino}(oxo)acetic acid
([2-(3-chlorophenyl)ethyl]{4-[(1Z)-dec-1-enyl]benzyl}amino)(oxo)acetic acid
{[2-(3-chlorophenyl)ethyl][4-(3-undecyl-1,2,4-oxadiazol-5-yl)benzyl]amino}(oxo)acetic acid
oxo {{(1R)-1-[4-(trifluoromethyl)phenyl]ethyl}[4-(3-undecyl-1,2,4-oxadiazol-5-yl)benzyl]-amino}acetic acid
oxo {[4-(trifluoromethyl)phenyl][4-(3-undecyl-1,2,4-oxadiazol-5-yl)benzyl]amino}acetic acid
oxo {{(1S)-1-[4-(trifluoromethyl)phenyl]ethyl}[4-(3-undecyl-1,2,4-oxadiazol-5-yl)benzyl]-amino}acetic acid
((4-dec-1-ynylbenzyl){1-[4-(trifluoromethyl)phenyl]ethyl}amino)(oxo)acetic acid
{{1-methyl-1-[4-(trifluoromethyl)phenyl]ethyl}[4-(3-undecyl-1,2,4-oxadiazol-5-yl)-benzyl]amino}(oxo)acetic acid
{{[4-(dodecyloxy)-1-naphthyl]methyl}[4-(trifluoromethyl)benzyl]amino}(oxo)acetic acid
((4-dec-1-ynylbenzyl){1-methyl-1-[4-(trifluoromethyl)phenyl]ethyl}amino)(oxo)acetic acid
{(4-dec-1-ynylbenzyl)[4-(trifluoromethyl)benzyl]amino}(oxo)acetic acid
oxo {[4-(trifluoromethyl)benzyl][3-(3-undecyl-1,2,4-oxadiazol-5-yl)benzyl]amino}acetic acid
{(4-dodecylbenzyl)[4-(trifluoromethyl)benzyl]amino}(oxo)acetic acid
{(3,5-dichlorobenzyl)[4-(tridecanoylamino)benzyl]amino}(oxo)acetic acid
{{4-[(4-octylphenyl)ethynyl]benzyl}[4-(trifluoromethyl)benzyl]amino}(oxo)acetic acid
oxo {[4-(trifluoromethyl)benzyl][4-(5-undecyl-1,2,4-oxadiazol-3-yl)benzyl]amino}acetic acid
{{4-[2-(4-octylphenyl)ethyl]benzyl}[4-(trifluoromethyl)benzyl]amino}(oxo)acetic acid
{(4-{[4-(heptyloxy)phenyl]ethynyl}benzyl)[4-(trifluoromethyl)benzyl]amino}(oxo)acetic acid
{{4-[(4-butylphenyl)ethynyl]benzyl}[4-(trifluoromethyl)benzyl]amino}(oxo)acetic acid
[[2-(3-chlorophenyl)ethyl](4-dodec-1-ynylbenzyl)amino](oxo)acetic acid
{(4-{[4-(benzyloxy)phenyl]ethynyl}benzyl)[4-(trifluoromethyl)benzyl]amino}(oxo)acetic acid
{{4-[2-(4-hexylphenyl)ethyl]benzyl}[4-(trifluoromethyl)benzyl]amino}(oxo)acetic acid
{{4-[2-(4-hexylphenyl)ethyl]benzyl}[4-(trifluoromethyl)benzyl]amino}(oxo)acetic acid
{[4-(11-hydroxyundecyl)benzyl][4-(trifluoromethyl)benzyl]amino}(oxo)acetic acid
{(4-dodec-1-ynylbenzyl)[4-(trifluoromethyl)phenyl]amino}(oxo)acetic acid
{{4-[(4-octylbenzoyl)amino]benzyl}[4-(trifluoromethyl)benzyl]amino}(oxo)acetic acid
{[(3-dec-1-ynyl-1-benzofuran-5-yl)methyl][4-(trifluoromethyl)benzyl]amino}(oxo)acetic acid
{[(3-dodec-1-ynyl-1-benzofuran-5-yl)methyl][4-(trifluoromethyl)benzyl]amino}(oxo)acetic acid
oxo {({3-[(4-propylphenyl)ethynyl]-1-benzofuran-5-yl}methyl)[4-(trifluoromethyl)benzyl]-amino}acetic acid
[(4-dodec-1-ynylbenzyl)(4-fluorobenzyl)amino](oxo)acetic acid
[bis(4-oct-1-ynylbenzyl)amino](oxo)acetic acid
{[(6-dodec-1-ynylpyridin-3-yl)methyl][4-(trifluoromethyl)benzyl]amino}(oxo)acetic acid
{[(4-dodec-1-ynyl-1-naphthyl)methyl][4-(trifluoromethyl)benzyl]amino}(oxo)acetic acid
([1-(3-chlorophenyl)-1-methylethyl]{4-[(4-hexylphenyl)ethynyl]benzyl}amino)(oxo)acetic acid
oxo {[4-(trifluoromethyl)benzyl][4-(4-undecyl-1,3-thiazol-2-yl)benzyl]amino}acetic acid
({4-[(4-hexylphenyl)ethynyl]benzyl}{1-methyl-1-[4-(trifluoromethyl)phenyl]ethyl}amino)-(oxo)acetic acid
{[4-(5-cyclohexylpent-1-ynyl)benzyl][4-(trifluoromethyl)benzyl]amino}(oxo)acetic acid
{{3-[(4-hexylphenyl)ethynyl]benzyl}[4-(trifluoromethyl)benzyl]amino}(oxo)acetic acid
{(4-dec-1-ynylbenzyl)[4-(trifluoromethyl)benzyl]amino}(oxo)acetic acid

Further Glepp-1 inhibitors which are not specifically disclosed in WO 03/064376 are:
{(4-dodec-1-yn-1-ylphenyl)[4-(trifluoromethyl)benzyl]amino}(oxo)acetic acid
((3-chlorobenzyl){4-[(4-hexylphenyl)ethynyl]benzyl}amino)(oxo)acetic acid
{{4-[(4-hexylphenyl)ethynyl]benzyl}[2-(trifluoromethyl)benzyl]amino}(oxo)acetic acid
((4-chlorobenzyl){4-[(4-hexylphenyl)ethynyl]benzyl}amino)(oxo)acetic acid
{{2-[(4-hexylphenyl)ethynyl]benzyl}[4-(trifluoromethyl)benzyl]amino}(oxo)acetic acid
{[4-(11-fluoroundec-1-yn-1-yl)benzyl][4-(trifluoromethyl)benzyl]amino}(oxo)acetic acid
{(4-non-1-yn-1-yl)benzyl)[4-(trifluoromethyl)benzyl]amino}(oxo)acetic acid
({4-[(4-butylphenyl)ethynyl]benzyl}{1-methyl-1-[4-(trifluoromethyl)phenyl]-ethyl}amino)(oxo)acetic acid
[{4-[(4-butylphenyl)ethynyl]benzyl}(diphenylmethyl)amino](oxo)acetic acid
{{4-[(4-hexylphenyl)ethynyl]phenyl}[4-(trifluoromethyl)benzyl]amino}(oxo)acetic acid
oxo {{4-[(4-pentylphenyl)ethynyl]benzyl}[4-(trifluoromethyl)benzyl]amino}acetic acid
{{4-[(4-heptylphenyl)ethynyl]benzyl}[4-(trifluoromethyl)benzyl]amino}(oxo)acetic acid
5-[(carboxycarbonyl)(4-dec-1-yn-1-ylbenzyl)amino]-2-hydroxybenzoic acid

Still further Glepp-1 inhibitors may be selected from Formula (VI)

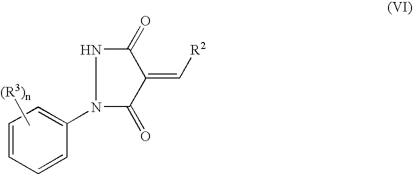

as well as its geometrical isomers, its optically active forms as enantiomers, diastereomers and its racemate forms, as well as pharmaceutically acceptable salts and pharmaceutically active derivatives thereof.

Compounds according to Formula (VI) as well as their manufacture are described in WO 02/102359.

In Formula (VI), R² is an unsubstituted or substituted phenyl which is fused with an aryl or an heteroaryl, or R² is an unsubstituted or substituted thienyl or unsubstituted or substituted furanyl, while R³ is iodine or unsubstituted or substituted phenyl, n is a integer of 0-4, preferably 1.

Still further Glepp-1 inhibitors may be selected from Formula (VII)

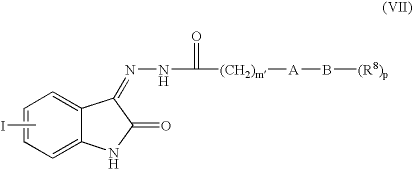

as well as its geometrical isomers, its optically active forms as enantiomers, diastereomers and its racemate forms, as well as pharmaceutically acceptable salts and pharmaceutically active derivatives thereof.

Compounds according to Formula (VII) as well as their manufacture is described in WO 03/037328.

In Formula (VII), m′ is 0, 1 or 2 and p is an integer from 1 to 3.

A is O, or a bond, B is selected from the group consisting of substituted or unsubstituted arylene, substituted or unsubstituted heteroarylene, substituted or unsubstituted heterocycloalkylene or substituted or unsubstituted cycloalkylene, further B is substituted or unsubstituted a phenyl group.

R⁸ is selected from the group comprising or consisting of H, halogen, hydroxy, acyl, amino, carboxy, cyano, nitro, an unsubstituted or substituted C₁-C₆-alkyl, an unsubstituted or substituted C₂-C₆-alkenyl, an unsubstituted or substituted C₂-C₆-alkynyl, an unsubstituted or substituted C₁-C₆-alkyl carboxy, an unsubstituted or substituted C₁-C₆-alkyl acyl, an unsubstituted or substituted C₁-C₆-alkyl alkoxycarbonyl, an unsubstituted or substituted C₁-C₆-alkyl aminocarbonyl, an unsubstituted or substituted C₁-C₆-alkyl acyloxy, acylamino, an unsubstituted or substituted C₁-C₆-alkyl acylamino, ureido, an unsubstituted or substituted C₁-C₆-alkyl ureido, an unsubstituted or substituted C₁-C₆-alkyl carbamate, an unsubstituted or substituted C₁-C₆-alkyl amino, C₁-C₆ alkoxy, an unsubstituted or substituted C₁-C₆-alkyl alkoxy, sulfanyl, an unsubstituted or substituted C₁-C₆-alkyl sulfanyl, sulfinyl, an unsubstituted or substituted C₁-C₆-alkyl sulfinyl, sulfonyl, sulfonylamino, an unsubstituted or substituted C₁-C₆-alkyl sulfonyl, an unsubstituted or substituted C₁-C₆-alkyl sulfonylaminoaryl, substituted or unsubstituted aryl, substituted or unsubstituted heteroaryl, an unsubstituted or substituted C₃-C₈-cycloalkyl or heterocycloalkyl, an unsubstituted or substituted C₁-C₆-alkyl aryl, an unsubstituted or substituted C₁-C₆-alkyl heteroaryl, an unsubstituted or substituted C₂-C₆-alkenyl-aryl or -heteroaryl, an unsubstituted or substituted C₂-C₆-alkynyl aryl or -heteroaryl.

A further aspect of the present invention consists in the use of a Glepp-1 inhibitor for the treatment of autoimmune and/or inflammatory disorders.

Still a further aspect of the present invention consists in Glepp-1 inhibitors for the treatment of autoimmune and/or inflammatory disorders.

Glepp-inhibitors may be identified by an assay methods described below (The Glepp-1 Binding Assay (in vitro assay)).

When employed as pharmaceuticals, Glepp-1 inhibitors of the present invention are typically administered in the form of a pharmaceutical composition. Hence, pharmaceutical compositions comprising a compound of Formula (I), (II), (III), (IV), (V), (VI) and (VII) and a pharmaceutically acceptable carrier, diluent or excipient therefore are also within the scope of the present invention. A person skilled in the art is aware of a whole variety of such carrier, diluent or excipient compounds suitable to formulate a pharmaceutical composition.

Such compositions can be prepared in a manner well known in the pharmaceutical art and comprise at least one active compound. Generally, the compounds of this invention are administered in a pharmaceutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The compounds of the invention, together with a conventionally employed adjuvant, car-rier, diluent or excipient may be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, or in the form of sterile injectable solutions for parenteral (including subcutaneous use). Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed.

The pharmaceutical compositions of these inventions can be administered by a variety of routes including oral, rectal, transdermal, subcutaneous, intravenous, intramuscular, and intranasal. The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, the carboxylic acid according to the invention is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatine; an excipient such as starch or lactose, a disintegrating agent such as alginic acid, Primogel, or corn starch; a lubricant such as magnesium stearate; a glidant such as colloidal silicon dio-xide; a sweetening agent such as sucrose or saccharin; or a flavoring agent such as pepper-mint, methyl salicylate, or orange flavoring.

Injectable compositions are typically based upon injectable sterile saline or phosphate-buf-fered saline or other injectable carriers known in the art. As above mentioned, compounds of Formula (I), (II), (III), (IV), (V), (VI) and (VII) in such compositions is typically a minor component, frequently ranging between 0.05 to 10% by weight with the remainder being the injectable carrier and the like.

The above described components for orally administered or injectable compositions are merely representative. Further materials as well as processing techniques and the like are set out in Part 5 of Remington's Pharmaceutical Sciences, 20^{th} Edition, 2000, Marck Publishing Company, Easton, Pa., which is incorporated herein be reference.

The compounds of this invention can also be administered in sustained release forms or from sustained release drug delivery systems. A description of representative sustained release materials can also be found in the incorporated materials in Remington's Pharma-ceutical Sciences.

### EXAMPLE 1

### Biological Assays

In the following an assay shall be presented that allow the identification of Glepp-1 inhibitors. Also, example assays in support of the anti-inflammatory effect of Glepp-1 inhibitors shall be presented.

The compounds of Formula (I), (II), (III), (IV), (V), (VI) and (VII), may be subjected to the following assays:
(1) Glepp-1 binding assay (in vitro)
(2) In vitro assay(s): Cell functional effect. Effect on MCP-1-induced chemotaxis on THP-1 cells
(3) Lymphocyte recruitment by thioglycollate (in vivo model for inflammation)
(4) Collagen induced arthritis (CIA) in mice (in vivo model for inflammation)
(5) Contact hypersensitivity (CHS is a T cell-mediated model of skin inflammation)
(6) EAE (in vivo model for MS)
(7) Ulcerative colitis

### (1) The Glepp-1 Binding Assay (In Vitro Assay)

Assays for the determination of the PTP inhibitory activity of test compounds are well known to a person skilled in the art. An example of such an assay is described below:

The PTP Enzyme Assay aims at determining the extent of inhibition of Glepp-1 in the presence of a test compound. The inhibition is illustrated by IC₅₀ values which denote the concentration of test compound necessary to achieve an inhibition of 50% of Glepp-1 using the following concentration of the Glepp-1 substrate DiFMUP:

### −μM DiFMUP for GLEPP-1.

### a) PTPs Cloning

The cloning and expression of the catalytic domain e.g. of Glepp-1, may be performed as described in S. Wächli et al.: J. Biol. Chem. 2000, 275(13), pp 9792-9796.

### b) Materials and Methods

The DiFMUP assay allows to follow the dephosphorylation of DiFMUP (6,8-DiFluoro-4-MethylUmbelliferyl Phosphate)—which is the Glepp-1 substrate—mediated by Glepp-1 into its stable hydrolysis product, i.e. DiFMU (6,8-difluoro-7-hydroxy coumarin). Due to its rather low pKa and its high quantum yield, DiFMU allows to measure both acidic and alkaline phosphatase activities with a great sensitivity.

Assays were performed in a 96 well plate format, using the catalytic core of a human recombinant Glepp-1 as the enzyme and 6,8-DiFluoro-4-MethylUmbelliferyl Phosphate (DiFMUP, Molecular Probes, D-6567) as a substrate. Compounds to be tested were dissolved in 100% DMSO at a concentration of 2 mM. Subsequent dilutions of the test compounds (to yield a concentration of 100, 30, 10, 3, 1, 0.3, 0.1, 0.03, 0.01, 0.001 μM) were performed in 60% DMSO manually. 8 μl of diluted compound or vehicle (60% DMSO=control) was distributed to a black Costar 96 well plate. 42 μl of human recombinant Glepp-1 enzyme diluted in assay buffer (20 mM Tris HCl pH 7.5, 0.01% IGEPAL CA-630, 0.1 mM ethylenediaminetetracetic acid, 1 mM DL-Dithiothreitol) can be added to the dilutions of compound or vehicle (distributed to a black Costar 96 well plate), followed by 50 μl of DiFMUP diluted in the assay buffer. The reaction ran for 30 minutes at room temperature before reading the fluorescence intensity (integral or intensity) on a Perkin-Elmer Victor 2 spectrofluorimeter (excitation of 6,8-difluoro-7-hydroxy coumarin is at 355 nm, the emission at 460 nm, for 0.1 s). The percentage of inhibition is determined by measuring the relative fluorescence ion absence of a test compound (PTP inhibitor), i.e. with the solvent alone (5% DMSO). The IC₅₀ values for inhibition were determined in triplicates.

The tested compounds according to Formula (I) display an inhibition (illustrated by IC₅₀ values) with regard to Glepp-1 of preferably less than 20 μM, more preferred less than 5 μM.

TABLE 1
IC₅₀ values for test compounds (Glepp-1 Inhibitors)

| | Inhibition |
|---|---|
| Glepp-1 Inhibitor (Test Compound) | (IC₅₀) |
| (4E)-4-[(5-bromo-2-furyl)methylene]-1-(4- | <1 μM |
| iodophenyl)pyrazolidine-3,5-dione | |
| 4-cyano-N-(4-{[(2Z)-2-(5-iodo-2-oxo-1,2- | <1 μM |
| dihydro-3H-indol-3- | |
| ylidene)hydrazino]carbonyl}phenyl)benzamide | |
| ({{4-[(4-hexylphenyl)ethynyl]benzyl}[4- | <1 μM |
| (trifluoromethyl)benzyl]-amino}- | |
| (oxo)acetic acid | |
| 5-((4-dec-1-ynylbenzyl){(2E)-3-[3- | <1 μM |
| (trifluoromethyl)phenyl]prop-2- | |
| enoyl}amino)-2-hydroxybenzoic acid | |
| (4-{[[(2-{4-[(octylamino)carbonyl]phenyl}- | <1 μM |
| 1,3-thiazol-4-yl)methyl](3- | |
| phenylpropanoyl)amino]methyl}phenoxy)acetic acid | |
| 5-[{4-[(4-butylphenyl)ethynyl]benzyl}(hexyl)amino]- | <1 μM |
| 2-hydroxy-benzoic acid | |
| 5-{[{4-[(4- | <1 μM |
| butylphenyl)ethynyl]benzyl}(hexyl)amino]carbonyl}- | |
| 2-hydroxybenzoicacid | |

(2) In Vitro Assay(s): Cell Functional Effect. In Vitro Chemotaxis.

The assay was performed as follows: After starvation of THP1 cells in serum free medium for 3 hours, 10⁷ cells/ml in medium containing 0.5% BSA, and inhibitors, when indicated. 100 μl of cells were applied to the top chamber (5 μm pore size transwell plates, COSTAR, New York) and 6001 of medium containing MCP-1/CCL2, in the absence or presence of test compounds, were added to the lower chamber. After incubation at 37° C. and 5% of CO₂ for 3 hours, cells that passed through the membrane were collected and counted in a Beckman Coulter® AcT 5Diff™.

Test Compounds ({{4-[(4-hexylphenyl)ethynyl]benzyl}[4-(trifluoromethyl)-benzyl]amino}-(oxo)acetic acid, 5-[{4-[(4-butylphenyl)ethynyl]benzyl}(hexyl)amino]-2-hydroxybenzoic acid, 5-{[{4-[(4-butylphenyl)ethynyl]benzyl}(hexyl)amino]carbonyl}-2-hydroxybenzoic acid) block chemotaxis of lymphocytes with EC₅₀ between 1 and 15 uM.

In vitro chemotaxis using human monocytes:

The assay was performed as follows:

Material:
Medium (RPMI 1640 without phenol red), containing L-glutamine, 0.3% DMSO and 2% foetal calf serum.
96-well filter plates (Neuro Probe Inc, ChemoTX #101-5) with a 5 micron pore size.
96-well funnel plates (Neuro Probe Inc, FP1).
Black 96-well reader plates (Costar # 3915).
Chemokine (MCP-1, R&D systems), use at 1 micro molar.
CyQUANT Cell Proliferation Assay Kit (Molecular Probe Inc. # C7026).
Buffy coats for preparation of monocytes by MACS sorting.
autoMACS® purification kit (Miltenyi Biotec # 130-091-153).

Method:
Prepare monocytes from a single buffy coat by negative depletion using the autoMACS® magnetic cell sorting kit for preparation of ‘untouched’ monocytes.
Incubate monocytes (1×10⁶ cells/ml) with test compounds to chosen final concentration(s) for 20 minutes at 37° C.
Prepare chemotaxis plate: add chemokine to the lower chambers of the chemotaxis wells (during this time it is advisable to place the filter to one side, inside the plate cover, and to avoid touching the filter).
Add the test compounds, where appropriate, to the lower chambers of the chemotaxis wells with the chemokine (N.B. the compounds are thus present in both the upper and lower chambers during the chemotaxis).
Distribute the monocytes (20,000 cells per well in a total volume of 20 micro litres) on to the top of the plate filter taking care to avoid air bubbles. Replace plate lid.
Incubate at 37° C. for 2 hours.
Carefully remove the plate from the incubator and remove the lid. Wash the filter with 10 ml of PBS in order to remove cells that have not migrated. This is achieved by using a 10 ml pipette and inclining the plate at 45°. Without touching the filter itself, let the PBS run over the filter by tapping the pipette tip gently against the metal border.
Transfer the migrated cells from the transmigration plate to a black reader plate with the aid of the funnel assembly and by centrifuging at 2000 rpm for 2 minutes.
Place an adhesive plate cover onto the plates and leave at −80° C. overnight.
The next day, defrost the plates and add CyQUANT.
Read the plates in a Wallac Victor Fluorescent Counter (excitation at 480 nm, emission at 520 n.

### (3) Lymphocyte Recruitment by Thioglycollate (In Vivo Model for Inflammation)

The following assay aims at determining the anti-inflammatory effect of Glepp-1 inhibitors in a model of lymphocyte recruitment by a thioglycollate (i.e. an inflammatory agent) challenge of mice, in vivo.

The assay was performed as follows:

A total of 6 C*H mice (Breeding Janvier about 8-9 weeks; obtained from IFFACREDO, l'Arbreste, France) were used. 2 groups, each consisting of 6 animals were formed:
Group 1: The animals were administered (per os) a dose of 30 mg/kg of Dexamethasone (positive control).
Group 2: The animals were administered (per os) a dose of 30 mg/kg of Glepp-1 inhibitor solubilized in the vehicle.
Group 3: Negative control (vehicle)
Group 4: Sham or baseline: the animals, receive the vehicle of the GLEPP and saline (“vehicle” of Thioglycollate).

### Experimental Protocol:

8-9 weeks old female C₃H mice were fasted during 18 hours. 15 minutes prior the intraperitoneal injection of thioglycollate (1.5%, 40 ml/kg), the mice were treated orally with a Glepp-1 inhibitor. Control mice received CMC/Tween as vehicle (10 ml/kg). The mice were then sacrificed by CO₂ inhalation and the peritoneal cavity was washed two times with 5 ml of ice-cold PBS/1 mM EDTA. The lavages were done 4 hrs or 48 hrs after thioglycollate challenge to evaluate neutrophils or macrophages recruitment, respectively. The white blood cells (neutrophils, lymphocytes or macrophages) were counted using a Beckman Coulter® A^{C}T 5Diff™. Dexamethasone was used as reference drug.

TABLE 2
Reduction of Neutrophils, Lymphocytes and Macrophages in blood (mice) following treatment with Glepp-1 inhibitor

| | Animals | Decrease | | Decrease | | Decrease | |
|---|---|---|---|---|---|---|---|
| Test Compound | per | in blood | | in blood | | in blood | |
| Glepp-1 Inhibitor | Group | Neutrophils | ±SEM | lymphocytes | ±SEM | Macrophages | ±SEM |
| {{4-[(4-hexylphenyl)ethynyl]benzyl}[4- | 6 | 27 | 5 | 29 | 5 | 27 | 7 |
| (trifluoromethyl)benzyl]amino}-(oxo)acetic acid | | | | | | | |
| 5-[{4- | 6 | 44 | 4 | 47 | 4 | 50 | 6 |
| [(4-butylphenyl)ethynyl]benzyl}(hexyl)amino]- | | | | | | | |
| 2-hydroxybenzoic acid | | | | | | | |
| 5-{[{4- [(4- | 6 | 41 | 6 | 41 | 5 | 62 | 3 |
| butylphenyl)ethynyl]benzyl}(hexyl)amino]carbonyl}- | | | | | | | |
| 2-hydroxy-benzoic acid | | | | | | | |
| ([1-(3-chlorophenyl)-1-methylethyl]{4-[(4- | 6 | 25 | 6 | 39 | 4 | 52 | 12 |
| hexylphenyl)ethynyl]benzyl}amino)(oxo)acetic acid | | | | | | | |
| {[4-(11-fluoroundec-1-yn-1-yl)benzyl][4- | 6 | 16 | 19 | 24 | 12 | 36 | 8 |
| (trifluoromethyl)benzyl]amino}(oxo)acetic acid | | | | | | | |
| {{4-[(4-hexylphenyl)ethynyl]phenyl}[4- | 6 | 15 | 7 | 21 | 9 | 32 | 6 |
| (trifluoromethyl)benzyl]amino}(oxo)acetic acid | | | | | | | |
| 4-[{4-[(4-butylphenyl)ethynyl]benzyl}(hexyl)amino]- | 6 | 45 | 3 | 23 | 8 | 65 | 9 |
| 2-hydroxybenzoic acid | | | | | | | |
| 5-[{4-[(4-butylphenyl)ethynyl]benzyl}(cyclopentyl- | 6 | 36 | 9 | 23 | 4 | 37 | 4 |
| methyl)amino]-2-fluorobenzoic acid | | | | | | | |
| 2-fluoro-5-(hexyl{4-[(4- | 6 | −6 | 9 | 4 | 6 | 26 | 7 |
| propylphenyl)ethynyl]benzyl}amino)benzoic acid | | | | | | | |
| 5-[{4-[(4-tert- | 6 | 36 | 7 | 13 | 12 | 42 | 9 |
| butylphenyl)ethynyl]benzyl}(hexyl)amino]-2- | | | | | | | |
| fluorobenzoic acid | | | | | | | |
| 5-{[{4-[(4- | 6 | 28 | 6 | 8 | 8 | 32 | 2 |
| butylphenyl)ethynyl]phenyl}(hexyl)amino]methyl}- | | | | | | | |
| 2-fluorobenzoic acid | | | | | | | |

(SEM = Standard Error of the Mean)

### (4) Collagen Induced Arthritis (CIA) in Mice (In Vivo Model for Inflammation)

The following assay aims at determining the anti-inflammatory effect of Glepp-1 inhibitors in a model of collagen induced arthritis in mice, in vivo.

The assay was performed as follows:

Induction of chronic polyarthritis in certain rodent strains can be provoked by two injections of heterologous type II collagen (collagen-induced arthritis; CIA) when mixed with an adjuvant oil (1-3). Collagen-induced arthritis (CIA) is viewed as a validated animal model of rheumatoid arthritis. In this model, a chronic peripheral arthritis is elicited by intradermal injection of homologous or heterologous (bovine, chicken) type II collagen (CII) in adjuvant into rats or mice. In rodents, susceptibility to CIA has been shown to be primarily controlled by MHC genes. Evidence has accumulated that CIA is dependent upon T cell activation, including the T cell proliferative response to mouse CII in immunized mice, the successful adoptive transfer of the disease with immune spleen cells, and the resistance of athymic nude animals to the induction of the pathology (1-2).

Animals: Mice; Strain: DBA/1j (Charles River, Calco, Italy); Sex: Male; Age: 8 to 9 weeks.
Acclimatization: Minimum of five days in the laboratory animal house where the experiment will take place.
Housing and feeding: the mice will be kept under standard laboratory conditions (non specific pathogen free) with ad libitum food and water.

### Induction of CIA:

Murine type II CIA may be induced as described elsewhere (4-5). Positive control drugs such as dexamethazone and specific TNF-alpha inhibitors are equally effective in chicken and bovine type II C— induced arthritis. Briefly, chicken CII is dissolved in 0.01 M acetic acid at a concentration of 2 mg/ml by stirring overnight at 4° C. CFA is prepared by adding Mycobacterium tuberculosis H37Ra (Difco, Detroit, Mich.)) at a concentration of 5 mg/ml. Before injection CII are emulsified with an equal volume of CFA.

To induce CIA, the mice are injected intradermally at the base of the tail with 100 ul of an emulsion containing 100 ug of CII. On day 21, a second injection of CII in CFA is administered.

### Therapeutic Treatment

For the therapeutic part of the study 5 groups of mice are created with animals presenting a clinical score≧1 and that will be treated for 10 days as follows:

Group 1: test compound at 15 mg/kg p.o. twice a day
Group 2: test compound at 30 mg/kg p.o. twice a day
Group 3: test compound at 45 mg/kg p.o. twice a day
Group 4: mice treated with vehicle (0.5% Methocel A4M/0.25% Tween-20 in water)
Group 5: dexamethasone, 0.3 mg/kg as positive control group

Each group consists of 10 mice. Animals will be sacrificed after the last day of treatment.

Clinical assessment of CIA: Mice are evaluated daily for arthritis according to a macros-copic scoring system (3-4): 0=no signs of arthritis; 1=swelling and/or redness of the paw or 1 digit; 2=involvement of 2 joints; 3=involvement of >2 joints, and 4=severe arthritis of the entire paw and digits. An arthritis index is calculated for each mouse by summing the scores for the individual paws.

Clinical severity is determined by quantitating changes in paw volume. Plethysmometry will be used to quantitate volume of both hindpaws.

Assessment of arthritis damage: The mice are sacrificed on day 28 post immunization. Blood is collected by intracardiac puncture and paws and knees are removed and fixed in 10% formalin for histologic examination by an observer unaware of the treatment regime of the mice. Histologic changes are examined by microscopy of both formalin-fixed paws and knees. Paws will be trimmed placed in decalcifying solution for 24 hours, embedded in paraffin, sectioned at 5 mcm, stained with hematoxilin and eosin and studied using light microscopy (4-5).

The following morphologic criteria are used: 0=no damage; 1=edema; 2=presence of inflammatory cells, and 3=bone resorption (4-5)

The histologic assessment of articular cartilage erosion is performed by eosin hematoxylin staining using the same criteria for assessing with safranin staining, as described below.

Articular cartilage erosion: The assessment is based on a numerical score, increasing with the severity and extension of the lesion. These numbers refer to the overall assessment of the whole section:

0=no erosion; articular cartilage intact
1-2=localized articular cartilage erosion
3=more extended erosions of the articular cartilage
4=diffuse and massive destruction of the articular cartilage

The histological parameters are measured in all the groups of prophylactically-treated mice.

### Blood Sampling

In case of ameliorative findings during the clinical observations period, two hours after the last treatment, blood samples is collected from each animal (before the sacrifice) of the treated groups.

### (5) Contact Hypersensitivity (CHS is a T Cell-Mediated Model of Skin Inflammation)

The following assay aims at determining the anti-inflammatory effect of Glepp-1 inhibitors in a model of skin inflammation in mice, in vivo.

The assay was performed as follows:

Material:
Female Balb/C mice between 8 wks and 12 weeks of age.
Solvent: Acetone/olive oil mix (4:1)
Hapten: DNFB (Dinitrofluorobenzene, Sigma #D1529).
Sensitizing solution: 0.5% DNFB in solvent, freshly prepared.
Challenging solution: 0.2% DNFB in solvent, freshly prepared.
Caliper (Mitutoyo, Urdorf, Switzerland)
Isofluran

### Methods

### Day 0: Sensitization of CHS:

For the induction of CHS groups of 72 mice are painted on the shaved back once with 35 μl of 0.5% DNFB in acetone/olive oil (4:1). 6 mice serve as negative control (unsensitized animals)

### Day 5: Elicitation of CHS:

Sensitized and unsensitized control animals are challenged (CH) on day +5 by applying 10 μl of 0.2% DNFB in acetone/olive oil (4:1) to each side of the right ears. As control, the left ear will be painted with an identical amount of the hapten vehicle.
1. n=12 Dosed with 0.5% Methocel A4M/0.25% Tween-20 in dH₂O p.o. 1 h before CH
2. n=12 Dosed with 1 mg/kg of test compound p.o. 1 h before CH
3. n=12 Dose with 3 mg/kg of test compound p.o. 1 h before CH
4. n=12 Dose with 10 mg/kg of test compound p.o. 1 h before CH
5. n=12 Dose with 30 mg/kg of test compound p.o. 1 h before CH
6. n=12 Dose with 1 mg/kg of Methotrexate s.c. 1 hour before CH

Right ears are partly removed and fixed in formaline and partly fixed in liquid nitrogen (for cytokine determination).

### Day 6: Evaluation of Ear Swelling (Basic Read Out)

Ear thickness of both ears is measured at three point each, ear swelling is evaluated according to the following formula:

[(Tₙ−T₅) right ear]−[(Tₙ−T₅) left ear]

where Tₙ and T₅ represent values of averaged ear thickness at day n of investigation and at day 5 pre challenge, respectively.

### (6) EAE (In Vivo Model for MS)

The following assay aims at determining the anti-inflammatory effect of Glepp-1 inhibitors in a model of EAE, in vivo.

The assay was performed as follows:

Effect of preventive treatment of a GLEPP inhibitor on a mouse chronic EAE (experimental autoimmune encephalomyelitis).

Materials

Mice: C₅₇ BL/6N colony was supplied by Charles River Italia (Calco, Lecco, Italy).
With acclimation: At least 5 days before the study is initiated. In this period the animals will be observed daily to ascertain their fitness for the study.
Age of About 8-week old; 18-22 g.

### Housing:

10 animals/cage in air-conditioned rooms.
Temperature: 22° C.±2
Relative humidity: 55%±10
Air changes: about 15-20/hour filtered on HEPA 99.99%.
Light: 12 hour cycle (7 a.m.-7 p.m.)
Cage: Makrolon® cage 42.5×26.6×15 h each fitted with a stainless steel cover-feed rack. A grill is inserted on the cage bottom. The waste that drops through the grill onto the cage bottom is periodically disposed of.

### Study Design

The study will involve 5 groups of 13 animals each. All the groups are assigned to the following treatments:

| | Test | | | Administration | Administration | Treatment | |
|---|---|---|---|---|---|---|---|
| Group | Substance | Dose | | volume/rate | route | period | Frequency |
| 1 | Methocel | — | | 10 mL/kg | p.o. | 21 days | Bid |
| | 4C/Tween | | | | | | |
| | (0.25%) in | | | | | | |
| | water | | | | | | |
| 2 | Test | 3 | mg/kg | 10 mL/kg | p.o. | 21 days | Bid |
| | Compound | | | | | | |
| 3 | Test | 10 | mg/kg | 10 mL/kg | p.o. | 21 days | Bid |
| | Compound | | | | | | |
| 4 | Test | 30 | mg/kg | 10 mL/kg | p.o. | 21 days | Bid |
| | Compound | | | | | | |

Vehicle: Methocel 4C/Tween (0.25%) in water will be used to dilute the test compound (a Glepp inhibitor) to the appropriate concentration.
Administration route: A test compound (a Glepp inhibitor) at the dose of 3, 10 and 30 mg/kg will be administered p.o. in a volume of 10 mL/kg. Group 1 will be dosed p.o. with vehicle (Methocel 4C/Tween (0.25%) in water) in a volume of 10 mL/kg.

The curative treatment will start at day 7 post immunization and will be continued for 21 days.

### Clinical Observations:

Starting from day 7 post-immunization the animals will individually be examined for the presence of paralysis by means of a clinical score as follows:
0=no sign of disease
0.5=partial tail paralysis
1=tail paralysis
1.5=tail paralysis+partial unilateral hindlimb paralysis
2=tail paralysis+hindlimb weakness or partial hindlimb paralysis
2.5=tail paralysis+partial hindlimb paralysis (lowered pelvi)
3=tail paralysis+complete hindlimb paralysis
3.5=tail paralysis+complete hindlimb paralysis+incontinence
4=tail paralysis+hindlimb paralysis+weakness or partial paralysis of forelimbs
5=moribund or dead

### (7) Dextran Sodium Sulfate-Induced Ulcerative Colitis in Mice

The assay was performed as follows:

Ulcerative colitis (UC) was induced in female mice (Balb/c, 20-22 g, Elevage Janvier) by Dextran Sodium Sulfate (DSS 4%) administered in drinking water. The mice had free access to DSS during 7 days. Body weight was determined daily. The severity of the UC was assessed by a clinical score (range 0 to 4) estimating the constituency of the stool (0=firm, 1=loose, 2=diarrhea) and the presence of blood (0=no blood, 1=occult blood, 2=gross rectal bleeding). Seven days after the induction of the disease, the animals were sacrificed. The length and the weight of the colon were determined and the ratio Weight/Length/100 g body weight was calculated. The spleen weight was also determined.

Compound of the invention (10, 30 & 100 mg/kg, po) was suspended in 0.5% methocel/0.25% tween 20 and was administered twice daily at days 3, 4, 5 and 6 after the induction of the UC. Sulfasalazine (200 mg/kg, po) was used as reference compound.

### RESULTS

5-[{4-[(4-butylphenyl)ethynyl]benzyl}(hexyl)amino]-2-hydroxybenzoic acid (10, 30 & 100 mg/kg, po) prevented significantly the colon reduction, estimated by the Weight/Length ratio, (−22%*, −28%** & −35%***, respectively).

The splenomegaly was significantly reduced (−31%* & −65%***) and the clinical score was improved (−29% to −54% & −21% to −54%) when 5-[{4-[(4-butylphenyl)ethynyl]benzyl}-(hexyl)amino]-2-hydroxybenzoic acid was administered at the dose of 30 & 100 mg/kg, po, respectively.

The body weight loss was also prevented by 5-[{4-[(4-butylphenyl)ethynyl]-benzyl}(hexyl)amino]-2-hydroxybenzoic acid (10, 30 & 100 mg/kg, po).

5-{[{2-[(4-butylphenyl)ethynyl]benzyl}(hexyl)amino]carbonyl}-2-hydroxybenzoic acid (10& 30 mg/kg, po) prevented significantly the colon reduction, estimated by the Weight/Length ratio, (−47%***& −50%***, respectively).

The splenomegaly was significantly reduced (−25%* & −45%**) and the clinical score was improved (−9% to −29% & −14% to −29%) when 5-{[{2-[(4-butylphenyl)ethynyl]benzyl}(hexyl)amino]carbonyl}-2-hydroxybenzoic acid was administered at the dose of 10 & 30 mg/kg, po, respectively.

The body weight loss was also prevented by 5-{[{2-[(4-butylphenyl)ethynyl]benzyl}(hexyl)amino]carbonyl}-2-hydroxybenzoic acid (10 & 30 mg/kg, po).

*: p<0.05, **: p<0.01, ***: p<0.001 (drug treated groups versus vehicle treated group).

### LIST OF REFERENCES

Aguiar, R. C., Y. Yakushijin, S. Kharbanda, S. Tiwari, G. J. Freeman and M. A. Shipp (1999). “PTPROt: an alternatively spliced and developmentally regulated B-lymphoid phosphatase that promotes G0/G1 arrest.” Blood 94(7): 2403-13.
Boissier M C et al. Biphasic effect of interferon-gamma in murine collagen-induced arthritis. Eur. J. Immunol., 25: 1184-1190, 1995
Cuzzocrea S. et al. Reduction in the evolution of murine type II collagen-induced arthritis by treatment with rosiglitazone, a ligand of the peroxisome proliferator-activated receptor \tilde{γ}. Arthr. And Rheum., 48: 3544-3556, 2003
Harris E D Jr. Rheumatoid arthritis: pathophysiology and implications for therapy. N. Engl. J. Med., 322: 1277-1289, 1990
Hom J T et al. Interleukin-1 mediated acceleration of type II collagen-induced arthritis: effects of anti-inflammatory or anti-arthritic drugs. Agents Action, 33: 300-309, 1991
Mori, Y., J. Yin, F. Sato, A. Sterian, L. A. Simms, F. M. Selaru, K. Schulmann, Y. Xu, A. Olaru, S. Wang, E. Deacu, J. M. Abraham, J. Young, B. A. Leggett and S. J. Meltzer (2004). “Identification of genes uniquely involved infrequent microsatellite instability colon carcinogenesis by expression profiling combined with epigenetic scanning.” Cancer Res. 64(7): 2434-8.
Motiwala, T., H. Kutay, K. Ghoshal, S. Bai, H. Seimiya, T. Tsuruo, S. Suster, C. Morrison and S. T. Jacob (2004). “Protein tyrosinephosphatase receptor-type O(PTPRO) exhibits characteristics of a candidate tumor suppressor in human lung cancer.” Proc. Natl. Acad. Sci. U.S.A. 101(38): 13844-9.
Niels Peter Hundahl Moller et al. Current Opinion in Drug Discovery & Development 3(5), 527-540 (2000)
Pixley, F. J., P. S. Lee, J. S. Condeelis and E. R. Stanley (2001). “Protein tyrosine phosphatase phi regulates paxillin tyrosine phosphorylation and mediates colony-stimulating factor 1-induced morphological changes in macrophages.” Mol. Cell. Biol. 21(5): 1795-809.
Pixley, F. J., P. S. Lee, M. G. Dominguez, D. B. Einstein and E. R. Stanley (1995). “A heteromorphic protein-tyrosine phosphatase, PTP phi, is regulated by CSF-1 in macrophages.” J. Biol. Chem. 270(45): 27339-47.
S. Wäichli et al.: J. Biol. Chem. 2000, 275(13), pp 9792-9796
Seimiya, H. and T. Tsuruo (1993). “Differential expression of protein tyrosine phosphatase genes during phorbol ester-induced differentiation of human leukemia U937 cells.” Cell Growth Differ. 4(12): 1033-9.
Stuart J M. et al., Collagen autoimmune arthritis. Annu. Rev. Immunol., 2: 199-218, 1984
Tomas Mustelin et al, Nature, Vol. 5, page 43, January 2005
Wharram, B. L., M. Goyal, P. J. Gillespie, J. E. Wiggins, D. B. Kershaw, L. B. Holzman, R.
C. Dysko, T. L. Saunders, L. C. Samuelson and R. C. Wiggins (2000). “Alteredpodocyte structure in GLEPP1 (Ptpro)-deficient mice associated with hypertension and low glomerular filtration rate.” J. Clin. Invest. 106(10): 1281-90.
WO 02/102359
WO 03/037328
WO 2005/011685
WO 2005/012280
WO 2005/09773
WO03/064376

1-26. (canceled)

27. A method of treating an autoimmune or inflammatory disease or disorder comprising the inhibition of Glepp-1 with a composition comprising a Glepp-1 inhibitor of Formula (I): 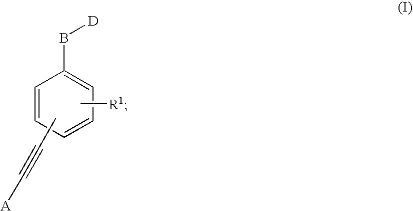
its geometrical isomers, optically active forms or its racemic forms, wherein:
A is selected from the group consisting of C₁-C₁₂-alkyl, C₁-C₈ alkyl, C₂-C₆-alkenyl, C₂-C₆-alkynyl, C₁-C₆-alkyl amine, C₁-C₆-alkyl alkoxy, aryl, heteroaryl, saturated or unsaturated C₃-C₉-cycloalkyl, heterocycloalkyl, C₁-C₆-alkyl aryl, C₁-C₆-alkyl heteroaryl, C₂-C₆-alkenyl aryl, C₂-C₆-alkenyl heteroaryl, C₂-C₆-alkynyl aryl, C₂-C₆-alkynyl heteroaryl, C₁-C₆-alkyl cycloalkyl, C₁-C₆-alkyl heterocycloalkyl, C₂-C₆-alkenyl cycloalkyl, C₂-C₆-alkenyl heterocycloalkyl, C₂-C₆-alkynyl cycloalkyl, and C₂-C₆-alkynyl heterocycloalkyl;
R¹ is selected from the group consisting of H, C₁-C₆-alkyl, C₁-C₆-alkoxy and halogen;
B is any one of B1 through B22; 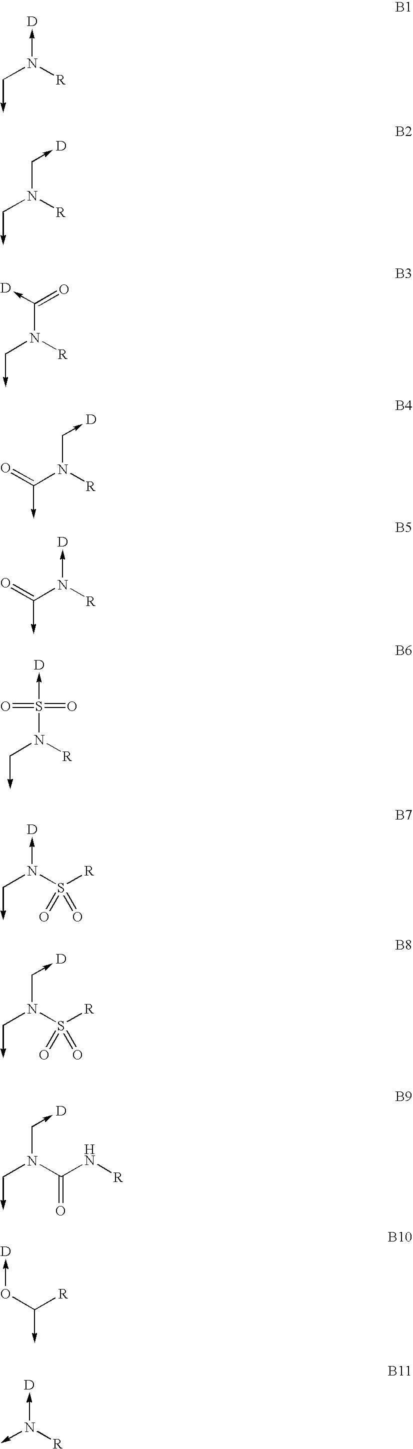 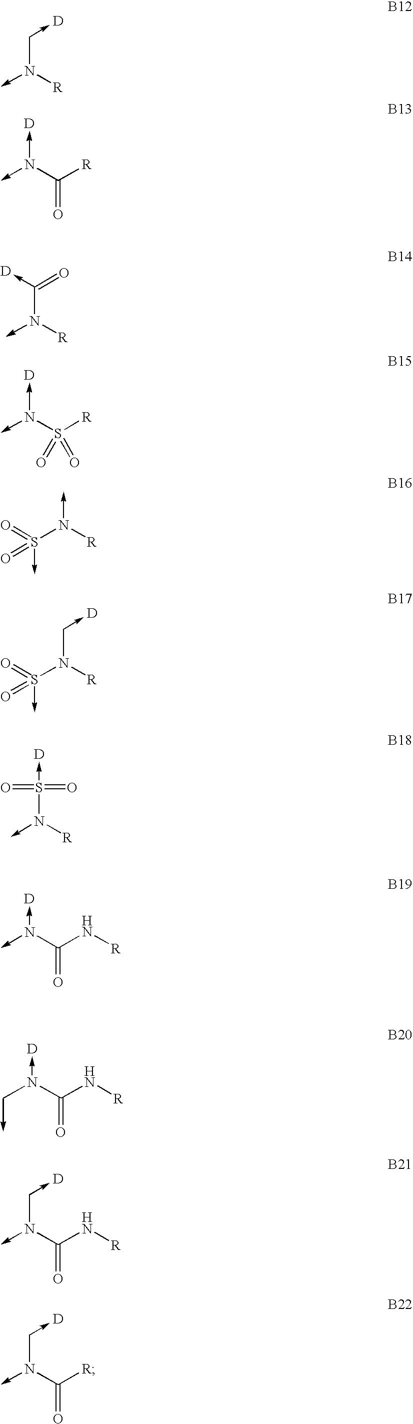
D is either selected from the group consisting of D1, D2 and D3: 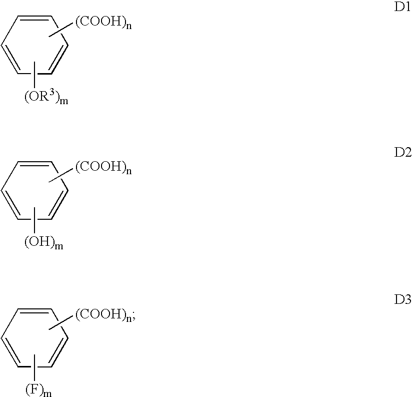
with m being an integer selected from 0, 1 or 2 and n being an integer selected from 1 or 2; and R³ is H or C₁-C₆ alkyl; or D4 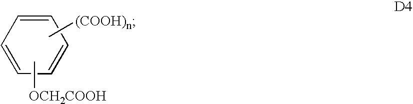
with n being an integer selected from 0 or 1;
R is selected from the group consisting of C₁-C₁₂-alkyl, C₁-C₈ alkyl, C₂-C₆-alkenyl, C₂-C₆-alkynyl, C₁-C₆-alkoxy, C₁-C₆-alkyl amine, C₁-C₆-alkyl alkoxy, aryl, heteroaryl, saturated or unsaturated C₃-C₈-cycloalkyl, heterocycloalkyl, C₁-C₆-alkyl aryl, C₁-C₆-alkyl heteroaryl, C₂-C₆-alkenyl aryl, C₂-C₆-alkenyl heteroaryl, C₂-C₆-alkynyl aryl, C₂-C₆-alkynyl heteroaryl, C₁-C₆-alkyl cycloalkyl, C₁-C₆-alkyl heterocycloalkyl, C₂-C₆-alkenyl cycloalkyl, C₂-C₆-alkenyl heterocycloalkyl, C₂-C₆-alkynyl cycloalkyl and C₂-C₆-alkynyl heterocycloalkyl.

28. The method according to claim 27, wherein the disease or disorder is selected from the group consisting of inflammatory bowel diseases, Crohn's disease, ulcerative colitis, collagenous colitis, lymphocytic colitis, diversion colitis irritable bowel syndrome, neuroinflammation, multiple sclerosis, Guillan Barre syndrome, chronic inflammatory polyneuropathy (CIPN), lung diseases, respiratory distress syndrome, joint and bone diseases, osteoarthritis, rheumatoid arthritis, liver diseases, liver fibrosis, cirrhosis, chronic liver disease, fibrotic diseases, lupus, glomerulosclerosis, systemic sclerosis skin fibrosis, post-radiation fibrosis, cystic fibrosis, vascular pathologies, atherosclerosis, cardiomyopathy, myocardial infarction, restenosis, degenerative diseases of the central nervous system, amyotrophic lateral sclerosis, inflammatory disorders of the skin, scleroderma and psoriasis.

29. The method according to claim 27, wherein the Glepp-1 inhibitor is a carboxylic acid of Formula (Ia):

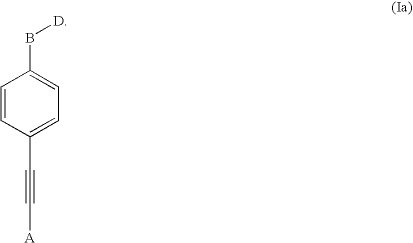

30. The method according to claim 27, wherein A is C₄-C₆-alkyl aryl or n-butylphenyl.

31. The method according to claim 27, wherein B is B1, B2, B3, B4, B5, B6, B7, B8, B9, B10, B12, B16, B17, B20 or B22.

32. The method according to claim 27, wherein R is a C₄-C₆-alkyl.

33. The method according to claim 27, wherein A is a phenyl group substituted by a C₁-C₄-alkyl or a halogen; B is either B1, B2, B3 or B12; R is C₄-C₆-alkyl, C₃-C₈-cycloalkyl or C₁-C₆-alkyl cycloalkyl; and D is selected from:

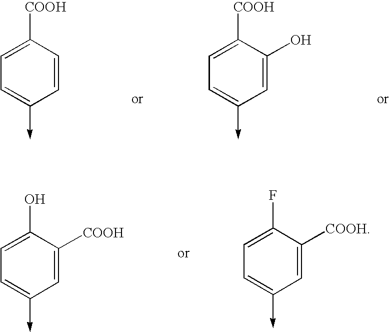

34. The method according to claim 27, wherein the Glepp-1 inhibitor is a carbolic acid of Formula (Ib) or Formula (Ic): 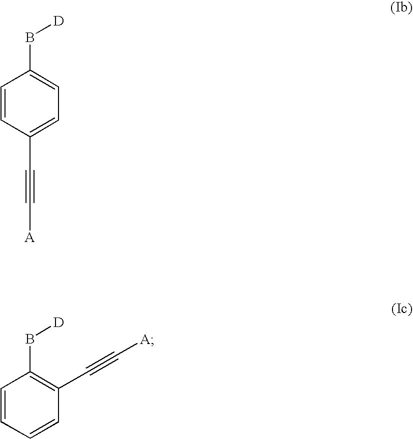
wherein A is a C₁-C₆-alkyl aryl or thiazolyl; and B is B1, B3, B4, B5, B11, B14 or B18.

35. The method according to claim 33, wherein the Glepp-1 inhibitor is selected from the group consisting of:
5-[{4-[(4-butylphenyl)ethynyl]benzyl}(hexyl)amino]-2-hydroxybenzoic acid;
5-[{4-[(4-butylphenyl)ethynyl]benzyl}(hexyl)amino]-2-fluorobenzoic acid;
4-({{4-[(4-butylphenyl)ethynyl]benzyl}[2-(4-chlorophenyl)ethyl]amino}-methyl)benzoic acid;
5-[{4-[(4-butylphenyl)ethynyl]benzyl}(3-phenylpropyl)amino]-2-hydroxybenzoic acid;
5-[{4-[(4-butylphenyl)ethynyl]benzyl}(1-naphthylmethyl)amino]-2-hydroxybenzoic acid;
5-((4-tert-butylbenzyl){4-[(4-butylphenyl)ethynyl]benzyl}amino)-2-hydroxybenzoic acid;
4-[{4-[(4-butylphenyl)ethynyl]benzyl}(hexyl)amino]-2-hydroxybenzoic acid;
2-fluoro-5-{hexyl[4-(phenylethynyl)benzyl]amino}benzoic acid;
5-[{4-[(4-chlorophenyl)ethynyl]benzyl}(hexyl)amino]-2-hydroxybenzoic acid;
5-(hexyl {4-[(4-methoxyphenyl)ethynyl]benzyl}amino)-2-hydroxybenzoic acid;
5-[hexyl(4-{[4-(trifluoromethyl)phenyl]ethynyl}benzyl)amino]-2-hydroxybenzoic acid;
5-[{4-[(4-butylphenyl)ethynyl]benzyl}(cyclopentylmethyl)amino]-2-fluorobenzoic acid;
5-[{4-[(4-butylphenyl)ethynyl]benzyl}(3,3-dimethylbutyl)amino]-2-fluorobenzoic acid;
5-((cyclopentylmethyl){4-[(4-methoxyphenyl)ethynyl]benzyl}amino)-2-hydroxybenzoic acid;
5-[{4-[(4-butylphenyl)ethynyl]benzyl}(ethyl)amino]-2-fluorobenzoic acid;
5-(hexyl {4-[(4-propylphenyl)ethynyl]benzyl}amino)-2-hydroxybenzoic acid;
5-[{4-[(4-butylphenyl)ethynyl]benzyl}(pentyl)amino]-2-fluorobenzoic acid;
5-[{4-[(4-butylphenyl)ethynyl]benzyl}(methyl)amino]-2-fluorobenzoic acid;
5-[{4-[(4-butylphenyl)ethynyl]benzyl}(cyclopropylmethyl)amino]-2-fluorobenzoic acid;
5-{butyl[4-(phenylethynyl)benzyl]amino}-2-fluorobenzoic acid;
2-fluoro-5-[[4-(phenylethynyl)benzyl](propyl)amino]benzoic acid;
2-fluoro-5-[{4-[(4-fluorophenyl)ethynyl]benzyl}(hexyl)amino]benzoic acid;
2-fluoro-5-(hexyl {4-[(4-propylphenyl)ethynyl]benzyl}amino)benzoic acid;
5-{{4-[(4-butylphenyl)ethynyl]benzyl}[(2-carboxycyclopropyl)methyl]amino}-2-fluorobenzoic acid;
5-[{4-[(4-ethylphenyl)ethynyl]benzyl}(hexyl)amino]-2-fluorobenzoic acid;
5-[{4-[(4-tert-butylphenyl)ethynyl]benzyl}(hexyl)amino]-2-fluorobenzoic acid;
5-{[{4-[(4-butylphenyl)ethynyl]phenyl}(hexyl)amino]methyl}-2-fluorobenzoic acid;
5-[{4-[(4-butylphenyl)ethynyl]benzyl}(isobutyl)amino]-2-fluorobenzoic acid;
5-[(1-{4-[(4-butylphenyl)ethynyl]phenyl}pentyl)oxy]-2-hydroxybenzoic acid;
5-{[{4-[(4-butylphenyl)ethynyl]benzyl}(hexyl)amino]carbonyl}-2-hydroxybenzoic acid;
5-{[{4-[(4-butylphenyl)ethynyl]benzoyl}(hexyl)amino]methyl}-2-hydroxybenzoic acid;
5-{[F {2-[(4-butylphenyl)ethynyl]benzyl}(hexyl)amino]carbonyl}-2-hydroxybenzoic acid;
4-((3-cyclopentylpropyl){{[(4-fluorophenyl)ethynyl]benzoyl}amino}-2-hydroxybenzoic acid;
4-[{4-[(4-butylphenyl)ethynyl]benzoyl}(3-cyclopentylpropyl)amino]-2-hydroxybenzoic acid;
5-{[{4-[(4-fluorophenyl)ethynyl]benzoyl}(hexyl)amino]methyl}-2-hydroxybenzoic acid;
5-{[{4-[(4-chlorophenyl)ethynyl]benzoyl}(hexyl)amino]methyl}-2-hydroxybenzoic acid;
5-{[{4-[(4-butylphenyl)ethynyl]benzyl}(hexyl)amino]carbonyl}-2-fluorobenzoic acid;
5-[{4-[(4-butylphenyl)ethynyl]benzoyl}(hexyl)amino]-2-fluorobenzoic acid;
5-{[{4-[(4-butylphenyl)ethynyl]benzoyl}(hexyl)amino]methyl}-2-fluorobenzoic acid; and
4-[{4-[(4-chlorophenyl)ethynyl]benzoyl}(3-cyclopentylpropyl)amino]-2-hydroxybenzoic acid.

36. The method according to claim 27, wherein the Glepp-1 inhibitor is an alkynyl aryl carboxamide according to Formula (II), which Formula (II) is selected from any of Formulae (IIa), (IIb), (IIc) or (IId): 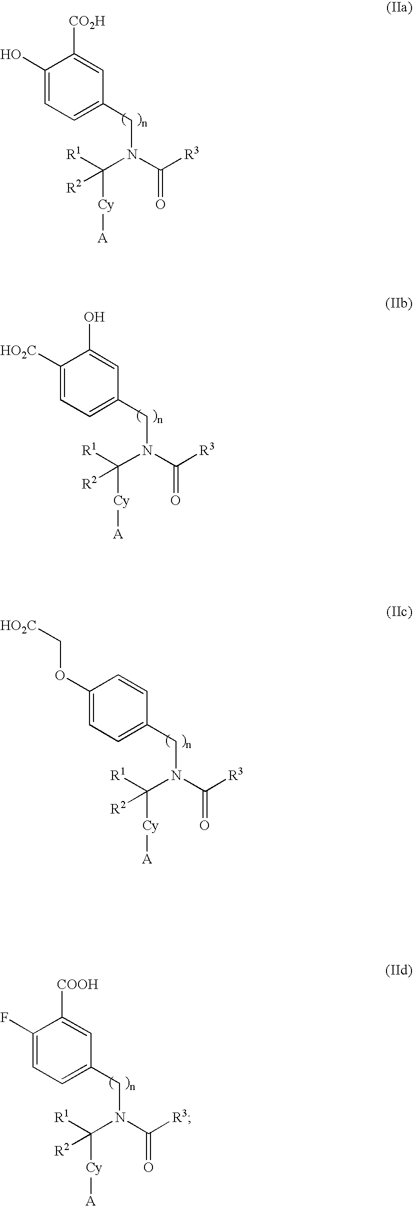
geometrical isomers, enantiomers, diastereomers and racemic forms thereof, wherein:
A is a C₂-C₁₅-alkynyl, C₂-C₆-alkynyl aryl, or C₂-C₆-alkynyl heteroaryl;
Cy is an aryl, an heteroaryl, a C₃-C₈-cycloalkyl or an heterocycle group; n is 0 or 1;
R¹ and R² are each independently selected from hydrogen or C₁-C₆-alkyl; and
R³ is selected from the group consisting of C₁-C₆-alkyl, C₂-C₆-alkenyl, C₂-C₆-alkynyl, C₁-C₆-alkoxy, C₁-C₆-alkyl amine, C₁-C₆-alkyl alkoxy, C₁-C₆-alkyl carboxy, aryl, heteroaryl, saturated or unsaturated C₃-C₈-cycloalkyl, heterocycloalkyl, acyl moiety, C₁-C₆-alkyl aryl, C₁-C₆-alkyl heteroaryl, C₂-C₆-alkenyl aryl, C₂-C₆-alkenyl heteroaryl, C₂-C₆-alkynyl aryl, C₂-C₆-alkynyl heteroaryl, C₁-C₆-alkyl cycloalkyl, C₁-C₆-alkyl heterocycloalkyl, C₂-C₆-alkenyl cycloalkyl, C₂-C₆-alkenyl heterocycloalkyl, C₂-C₆-alkynyl cycloalkyl, and C₂-C₆-alkynyl heterocycloalkyl.

37. The method according to claim 36, wherein R¹ and R² are each H, Cy is a phenyl group, and A is a moiety of the formula —C≡C—R⁶ wherein R⁶ is phenyl, C₁-C₁₂-alkyl phenyl, C₂-C₆-alkenyl phenyl, or C₂-C₆-alkynyl phenyl.

38. The method according to claim 37, wherein the Glepp-1 inhibitor is selected from the group consisting of:
4-[{4-[(4-butylphenyl)ethynyl]benzyl}(cyclopentylpropanoyl)amino]-2-hydroxy-benzoic acid;
5-[{4-[(4-butylphenyl)ethynyl]benzyl}(cyclohexylcarbonyl)amino]-2-hydroxybenzoic acid;
5-[{4-[(4-butylphenyl)ethynyl]benzyl}(hexanoyl)amino]2-hydroxybenzoic acid;
5-((4-tert-butylbenzoyl){4-[(4-butylphenyl)ethynyl]benzyl}amino)-2-hydroxybenzoic acid;
5-((biphenyl-4-ylcarbonyl){4-[(4-butylphenyl)ethynyl]benzyl}amino)-2-hydroxybenzoic acid;
5-[{4-[(4-butylphenyl)ethynyl]benzyl}(3,3-dimethylbutanoyl)amino]-2-hydroxybenzoic acid;
5-((1,3-benzodioxol-5-ylcarbonyl){4-[(4-butylphenyl)ethynyl]benzyl}amino)-2-hydroxybenzoic acid;
5-([(benzyloxy)acetyl]{4-[(4-butylphenyl)ethynyl]benzyl}amino)-2-hydroxybenzoic acid;
5-[{4-[(4-butylphenyl)ethynyl]benzyl}(4-hexylbenzoyl)amino]-2-hydroxybenzoic acid;
5-((1-benzothien-2-ylcarbonyl){4-[(4-butylphenyl)ethynyl]benzyl}amino)-2-hydroxybenzoic acid;
5-{[{4-[(4-butylphenyl)ethynyl]benzyl}(hexanoyl)amino]methyl}-2-hydroxybenzoic acid;
(4-{[{4-[(4-butylphenyl)ethynyl]benzyl}(hexanoyl)amino]methyl}phenoxy)acetic acid;
8-[{4-[(4-butylphenyl)ethynyl]benzyl}(3-cyclopentylpropanoyl)amino]-5,6,7,8-tetrahydronaphthalene-2-carboxylic acid;
4-{[{4-[(4-butylphenyl)ethynyl]benzyl}(3-cyclopentylpropanoyl)amino]methyl}-benzoic acid;
5-[{4-[(4-chlorophenyl)ethynyl]benzyl}(2-thienylacetyl)amino]-2-hydroxybenzoic acid;
5-[{4-[(4-chlorophenyl)ethynyl]benzyl}(2-thienylacetyl)amino]-2-hydroxybenzoic acid;
5-[{4-[(4-butylphenyl)ethynyl]benzyl}(3-cyclopentylpropanoyl)amino]-2-fluorobenzoic acid;
5-[{4-[(4-butylphenyl)ethynyl]benzyl}(3,3-dimethylbutanoyl)amino]-2-fluorobenzoic acid;
4-[{4-[(4-butylphenyl)ethynyl]benzyl}(cyclohexylcarbonyl)amino]-2-hydroxybenzoic acid;
4-[{4-[(4-butylphenyl)ethynyl]benzyl}(hexanoyl)amino]-2-hydroxybenzoic acid;
4-[{4-[(4-butylphenyl)ethynyl]benzyl}(3-cyclopentylpropanoyl)amino]-2-fluorobenzoic acid;
4-[{4-[(4-tert-butylphenyl)ethynyl]benzyl}(3-cyclopentylpropanoyl)amino]-2-hydroxybenzoic acid;
4-((3-cyclopentylpropanoyl){4-[(4-propoxyphenyl)ethynyl]benzyl}amino)-2-hydroxybenzoic acid;
4-((3-cyclopentylpropanoyl){4-[(4-propylphenyl)ethynyl]benzyl}amino)-2-hydroxybenzoic acid; and
5-{[{4-[(4-butylphenyl)ethynyl]benzyl}(cyclopentylpropanoyl)amino]methyl}-2-hydroxybenzoic acid.

39. The method according to claim 27, wherein the Glepp-1 inhibitor is an aryl dicarboxamide according to Formula (III), which Formula (III) is selected from any of the Formulae (IIIa), (IIIb) or (IIIc): 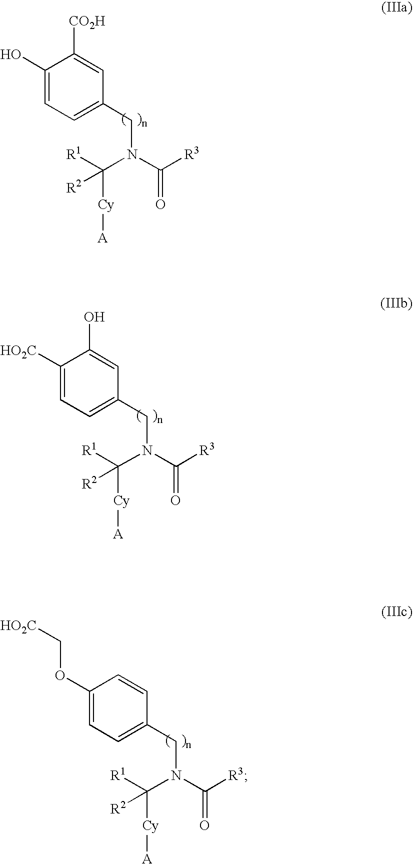
isomers, enantiomers, diastereomers or racemates thereof, wherein:
A is an aminocarbonyl moiety of the formula —CO—NHR⁶ wherein R⁶ is C₆-C₁₅-alkyl, C₂-C₁₅-alkenyl, C₂-C₁₅-alkynyl, a C₃-C₈-cycloalkyl, C₁-C₆ alkyl-C3-C8 cycloalkyl, phenyl, C₁-C₁₂-alkyl phenyl, C₂-C₆-alkenyl phenyl or C₂-C₆-alkynyl phenyl;
Cy is an aryl, heteroaryl, aryl-heteroaryl, heteroaryl-aryl, aryl-aryl, cycloalkyl or heterocycle group;
n is either 0 or 1;
R¹ and R² are each independently selected from hydrogen or C₁-C₆-alkyl; and
R³ is selected from the group consisting of C₁-C₆-alkyl, C₂-C₆-alkenyl, C₂-C₆-alkynyl, C₁-C₆-alkoxy, C₁-C₆-alkyl amine, C₁-C₆-alkyl alkoxy, aryl, heteroaryl, saturated or unsaturated C₃-C₈-cycloalkyl, heterocycloalkyl, acyl moiety, C₁-C₆-alkyl aryl, C₁-C₆-alkyl heteroaryl, C₂-C₆-alkenyl aryl, C₂-C₆-alkenyl heteroaryl, C₂-C₆-alkynyl aryl, C₂-C₆-alkynyl heteroaryl, C₁-C₆-alkyl cycloalkyl, C₁-C₆-alkyl heterocycloalkyl, C₂-C₆-alkenyl cycloalkyl, C₂-C₆-alkenyl heterocycloalkyl, C₂-C₆-alkynyl cycloalkyl and C₂-C₆-alkynyl heterocycloalkyl.

40. The method according to claim 39, wherein R¹ and R² are each H, Cy is selected from the group consisting of phenyl, thiazolyl, phenyl-thiazolyl and thiazolyl-phenyl and R⁶ is selected from the group consisting of C₈-C₁₂-alkyl, and C₁-C₄-alkyl phenyl which may be substituted by C₁-C₈-alkyl or phenoxy.

41. The method according to claim 40, wherein the Glepp-1 inhibitor is selected from the group consisting of:
5-[(3-cyclopentylpropanoyl)(4-{[(4-phenoxybenzyl)amino]carbonyl}-benzyl)amino]-2-hydroxybenzoic acid;
5-((4-cyanobenzoyl){[2-(4-{[(4-pentylbenzyl)amino]carbonyl}phenyl)-1,3-thiazol-4-yl]methyl}amino)-2-hydroxybenzoic acid;
(4-{[{[2-(4-{[(4-pentylbenzyl)amino]carbonyl}phenyl)-1,3-thiazol-4-yl]methyl}(3-phenylpropanoyl)amino]methyl}phenoxy)acetic acid; and
(4-{[[(2-{4-[(octylamino)carbonyl]phenyl}-1,3-thiazol-4-yl)methyl](3-phenylpropanoyl)amino]methyl}phenoxy)acetic acid.

42. The method according to claim 27, wherein the Glepp-1 inhibitor is a substituted methylene amide derivative of Formula (IV): 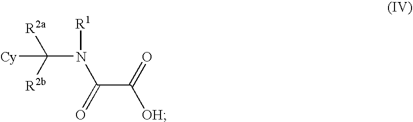
isomers, enantiomers, diastereomers, salts or racemates thereof, wherein:
R¹ is selected from C₁-C₁₂-alkyl, C₂-C₁₂-alkenyl, C₁-C₁₂-alkynyl, aryl, heteroaryl, C₃-C₈-cycloalkyl or heterocycloalkyl, C₁-C₁₂-alkyl-aryl or C₁-C₁₂-alkyl-heteroaryl, C₂-C₁₂-alkenyl-aryl or -heteroaryl, or C₂-C₁₂-alkynyl-aryl or -heteroaryl;
R^{2a} and R^{2b} are each independently selected from H or C₁-C₁₂-alkyl; and
Cy is an aryl, heteroaryl, cycloalkyl or heterocycle group.

43. The method according to claim 42, wherein:
R^{2a} and R^{2b} are each H;
R¹ is -CA₁A₂A₃ whereby each of A₁, A₂, A₃ is independently selected from the group consisting of hydrogen, methyl, ethyl, propyl, phenyl and thienyl, which may be substituted by cyano, halogen, methoxy, hydroxy, phenoxy, —NO₂ or trifluoromethyl; and Cy is a thienyl, phenyl or biphenyl being substituted by —SO₂R³ or —CO—NR³R^{3′} in which R^{3′} is H and R³ is C₇-C₁₅-alkyl or C₈-C₁₅-alkyl or a dodecyl group which may be substituted by a halogen, whereby however at least two of A₁, A₂, A₃ are not hydrogen, methyl, ethyl or propyl.

44. The method according to claim 43, wherein the Glepp-1 inhibitor has the Formula (V): 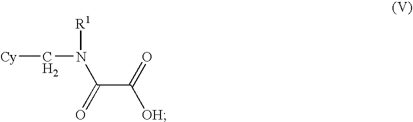
isomers, enantiomers, diastereomers, salts, and racemates thereof, wherein:
R¹ is selected from the group consisting of phenyl, benzyl, phenethyl, and 1-methylbenzyl which may be substituted by C₁-C₆-alkyl group or a cycloalkyl group;
Cy is a phenyl or a biphenyl group substituted with a moiety selected from the group consisting of —NH—CO—R³, —CO—NH—R³, and an oxadiazole group substituted with R³, wherein R³ is C₇-C₁₅-alkyl, C₈-C₁₅-alkyl or a dodecyl group.

45. The method according to claim 43, wherein the Glepp-1 inhibitor is selected from the group consisting of:
{{4-[(4-hexylphenyl)ethynyl]benzyl}[4-(trifluoromethyl)benzyl]amino}-(oxo)acetic acid;
(benzyl {4-[(pentadecylamino)carbonyl]benzyl}amino)(oxo)acetic acid;
({[1-(tert-butoxycarbonyl)-4-piperidinyl]methyl}{4-[(dodecylamino)carbonyl]-benzyl}amino)(oxo)acetic acid;
oxo {{4-[(9E)-9-tetradecenoylamino]benzyl}[4-(trifluoromethyl)benzyl]amino}acetic acid;
oxo {[4-(trifluoromethyl)benzyl][4-(3-undecyl-1,2,4-oxadiazol-5-yl)benzyl]amino}-acetic acid;
oxo {(4-tetradec-1-ynylbenzyl) [4-(trifluoromethyl)benzyl]amino}acetic acid;
{(4-dodec-1-ynylbenzyl)[4-(trifluoromethyl)benzyl]amino}(oxo)acetic acid;
oxo {{1-[4-(trifluoromethyl)phenyl]ethyl}[4-(3-undecyl-1,2,4-oxadiazol-5-yl)benzyl]amino}acetic acid;
([(2-butyl-1-benzofuran-3-yl)methyl]{4-[(dodecylamino)carbonyl]benzyl}amino)-(oxo)acetic acid;
{(4-{[(4-octylphenyl)amino]carbonyl}benzyl)[4-(trifluoromethyl)benzyl]amino}-(oxo)acetic acid;
{{cyclopentyl[4-(trifluoromethyl)phenyl]methyl}[4-(tridecanoylamino)benzyl]-amino}(oxo)acetic acid;
oxo([4-(trifluoromethyl)benzyl]{[4-(3-undecyl-1,2,4-oxadiazol-5-yl)-1-naphthyl]-methyl}amino)acetic acid;
{{cyclopentyl[4-(trifluoromethyl)phenyl]methyl}[4-(3-undecyl-1,2,4-oxadiazol-5-yl)benzyl]amino}(oxo)acetic acid;
([2-(3-chlorophenyl)ethyl]{4-[(1Z)-dec-1-enyl]benzyl}amino)(oxo)acetic acid;
{[2-(3-chlorophenyl)ethyl][4-(3-undecyl-1,2,4-oxadiazol-5-yl)benzyl]amino}-(oxo)acetic acid;
oxo {{(1R)-1-[4-(trifluoromethyl)phenyl]ethyl}[4-(3-undecyl-1,2,4-oxadiazol-5-yl)benzyl]amino}acetic acid;
oxo {[4-(trifluoromethyl)phenyl][4-(3-undecyl-1,2,4-oxadiazol-5-yl)benzyl]amino}-acetic acid;
oxo {{(1S)-1-[4-(trifluoromethyl)phenyl]ethyl}[4-(3-undecyl-1,2,4-oxadiazol-5-yl)benzyl]amino}acetic acid;
((4-dec-1-ynylbenzyl){1-[4-(trifluoromethyl)phenyl]ethyl}amino)(oxo)acetic acid;
{{1-methyl-1-[4-(trifluoromethyl)phenyl]ethyl}[4-(3-undecyl-1,2,4-oxadiazol-5-yl)benzyl]amino}(oxo)acetic acid;
{{[4-(dodecyloxy)-1-naphthyl]methyl}[4-(trifluoromethyl)benzyl]amino}(oxo)acetic acid;
((4-dec-1-ynylbenzyl){1-methyl-1-[4-(trifluoromethyl)phenyl]ethyl}amino)-(oxo)acetic acid;
{(4-dec-1-ynylbenzyl)[4 (trifluoromethyl)benzyl]amino}(oxo)acetic acid;
oxo {[4-(trifluoromethyl)benzyl][3-(3-undecyl-1,2,4-oxadiazol-5-yl)benzyl]-amino}acetic acid;
{(4-dodecylbenzyl)[4-(trifluoromethyl)benzyl]amino}(oxo)acetic acid;
{(3,5-dichlorobenzyl) [4-(tridecanoylamino)benzyl]amino}(oxo)acetic acid;
{{4-[(4-octylphenyl)ethynyl]benzyl}[4-(trifluoromethyl)benzyl]amino}(oxo)acetic acid;
oxo {[4-(trifluoromethyl)benzyl][4-(5-undecyl-1,2,4-oxadiazol-3-yl)benzyl]amino}-acetic acid;
{{4-[2-(4-octylphenyl)ethyl]benzyl}[4-(trifluoromethyl)benzyl]amino}(oxo)acetic acid;
{(4-{[4-(heptyloxy)phenyl]ethynyl}benzyl) [4-(trifluoromethyl)benzyl]amino}(oxo)-acetic acid;
{{4-[(4-butylphenyl)ethynyl]benzyl}[4-(trifluoromethyl)benzyl]amino}(oxo)acetic acid;
[[2-(3-chlorophenyl)ethyl](4-dodec-1-ynylbenzyl)amino](oxo)acetic acid;
{(4-{[4-(benzyloxy)phenyl]ethynyl}benzyl)[4-(trifluoromethyl)benzyl]amino}-(oxo)acetic acid;
{{4-[2-(4-hexylphenyl)ethyl]benzyl}[4 (trifluoromethyl)benzyl]amino}(oxo)acetic acid;
{{4-[2-(4-hexylphenyl)ethyl]benzyl}[4-(trifluoromethyl)benzyl]amino}(oxo)acetic acid;
{[4-(11-hydroxyundecyl)benzyl][4-(trifluoromethyl)benzyl]amino}(oxo)acetic acid;
{(4-dodec-1-ynylbenzyl) [4-(trifluoromethyl)phenyl]amino}(oxo)acetic acid;
{{4-[(4-octylbenzoyl)amino]benzyl}[4-(trifluoromethyl)benzyl]amino}(oxo)acetic acid;
{[(3-dec-1-ynyl-1-benzofuran-5-yl)methyl][4-(trifluoromethyl)benzyl]amino}-(oxo)acetic acid;
{[(3-dodec-1-ynyl-1-benzofuran-5-yl)methyl][4-(trifluoromethyl)benzyl]amino}-(oxo)acetic acid;
oxo {({3-[(4-propylphenyl)ethynyl]-1-benzofuran-5-yl}methyl) [4-(trifluoromethyl)-benzyl]amino}acetic acid;
[(4-dodec-1-ynylbenzyl)(4-fluorobenzyl)amino](oxo)acetic acid;
[bis(4-oct-1-ynylbenzyl)amino](oxo)acetic acid;
{[(6-dodec-1-ynylpyridin-3-yl)methyl][4-(trifluoromethyl)benzyl]amino}(oxo)acetic acid;
[(4-dodec-1-ynyl-1-naphthyl)methyl][4-(trifluoromethyl)benzyl]amino}(oxo)acetic acid;
([1-(3-chlorophenyl)-1-methylethyl]{4-[(4-hexylphenyl)ethynyl]benzyl}amino)-(oxo)acetic acid;
oxo {[4-(trifluoromethyl)benzyl][4-(4-undecyl-1,3-thiazol-2-yl)benzyl]amino}acetic acid;
({4-[(4-hexylphenyl)ethynyl]benzyl}{1-methyl-1-[4-(trifluoromethyl)phenyl]ethyl}-amino)(oxo)acetic acid;
{[4-(5-cyclohexylpent-1-ynyl)benzyl][4-(trifluoromethyl)benzyl]amino}(oxo)acetic acid;
{{3-[(4-hexylphenyl)ethynyl]benzyl}[4-(trifluoromethyl)benzyl]amino}(oxo)acetic acid;
{(4-dec-1-ynylbenzyl) [4-(trifluoromethyl)benzyl]amino}(oxo)acetic acid;
{(4-dodec-1-yn-1-ylphenyl)[4-(trifluoromethyl)benzyl]amino}(oxo)acetic acid;
((3-chlorobenzyl){4-[(4-hexylphenyl)ethynyl]benzyl}amino)(oxo)acetic acid;
{{4-[(4-hexylphenyl)ethynyl]benzyl}[2-(trifluoromethyl)benzyl]amino}(oxo)acetic acid;
((4-chlorobenzyl){4-[(4-hexylphenyl)ethynyl]benzyl}amino)(oxo)acetic acid;
{{2-[(4-hexylphenyl)ethynyl]benzyl}[4-(trifluoromethyl)benzyl]amino}(oxo)acetic acid;
{[4-(11-fluoroundec-1-yn-1-yl)benzyl][4-(trifluoromethyl)benzyl]amino}(oxo)acetic acid;
{(4-non-1-yn-1-ylbenzyl) [4-(trifluoromethyl)benzyl]amino}(oxo)acetic acid;
({4-[(4-butylphenyl)ethynyl]benzyl}{1-methyl-1-[4-(trifluoromethyl)phenyl]-ethyl}amino)(oxo)acetic acid;
[{4-[(4-butylphenyl)ethynyl]benzyl}(diphenylmethyl)amino](oxo)acetic acid;
{{4-[(4-hexylphenyl)ethynyl]phenyl}[4-(trifluoromethyl)benzyl]amino}(oxo)acetic acid;
oxo {{4-[(4-pentylphenyl)ethynyl]benzyl}[4-(trifluoromethyl)benzyl]amino}acetic acid;
{{4-[(4-heptylphenyl)ethynyl]benzyl}[4-(trifluoromethyl)benzyl]amino}(oxo)acetic acid; and
5-[(carboxycarbonyl)(4-dec-1-yn-1-ylbenzyl)amino]-2-hydroxybenzoic acid.

46. The method according to claim 27, wherein the Glepp-1 inhibitor is a compound of Formula (VI): 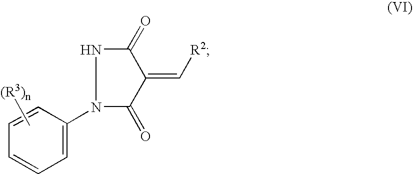
isomers, enantiomers, diastereomers, salts or racemates thereof, wherein:
R² is a phenyl which is fused with an aryl or an heteroaryl, or R² is a thienyl or furanyl, R³ is iodine or phenyl and n is a integer of 0-4.

47. The method according to claim 46, wherein n is 1.

48. The method according to claim 27, wherein the Glepp-1 inhibitor is a compound of Formula (VII): 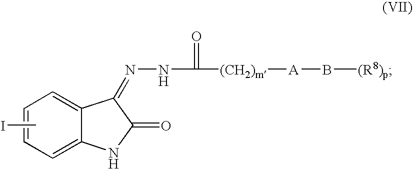
isomers, enantiomers, diastereomers, salts and racemates thereof, wherein:
m′ is 0, 1 or 2 and p is an integer from 1 to 3;
A is O, or a bond, B is arylene, heteroarylene, heterocycloalkylene, cycloalkylene or phenyl;
R⁸ is selected from the group comprising or consisting of H, halogen, hydroxy, acyl, amino, carboxy, cyano, nitro, C₁-C₆-alkyl, C₂-C₆-alkenyl, C₂-C₆-alkynyl, C₁-C₆-alkyl carboxy, C₁-C₆-alkyl acyl, C₁-C₆-alkyl alkoxycarbonyl, C₁-C₆-alkyl aminocarbonyl, C₁-C₆-alkyl acyloxy, acylamino, C₁-C₆-alkyl acylamino, ureido, C₁-C₆-alkyl ureido, C₁-C₆-alkyl carbamate, C₁-C₆-alkyl amino, C₁-C₆ alkoxy, C₁-C₆-alkyl alkoxy, sulfanyl, C₁-C₆-alkyl sulfanyl, sulfinyl, C₁-C₆-alkyl sulfinyl, sulfonyl, sulfonylamino, C₁-C₆-alkyl sulfonyl, C₁-C₆-alkyl sulfonylaminoaryl, aryl, heteroaryl, C₃-C₈-cycloalkyl or heterocycloalkyl, C₁-C₆-alkyl aryl, C₁-C₆-alkyl heteroaryl, C₂-C₆-alkenyl-aryl or -heteroaryl, and C₂-C₆-alkynyl aryl or -heteroaryl.

49. The method according to claim 27, wherein the Glepp-1 inhibitor is administered in combination with a co-agent useful in the treatment of an autoimmune and/or an inflammatory disorder.

50. A pharmaceutical composition comprising a Glepp-1 inhibitor or any one of Formulae I through VII, a co-agent useful in the treatment of an autoimmune and/or an inflammatory disorder and a pharmaceutically acceptable excipient.

51. The pharmaceutical composition according to claim 50, wherein the co-agent is interferon beta.
